(19) United States
(12) Patent Application Publication
(10) Pub. No.: US 2026/0350791 A1
(43) Pub. Date: Nov. 5, 2026

(54) APPARATUS, EVALUATION DEVICE, EVALUATION SYSTEM AND METHOD FOR A CONDITION ANALYSIS OF A PLANT

(71) Applicant: Carl Zeiss Spectroscopy GmbH, Jena (DE)

(72) Inventors: Alexander FREYTAG, Jena (DE); Guillaume GOMARD, Eggenstein-Leopoldshafen (DE); Karsten LINDIG, Jena (DE); Michael RODE, Jean (DE); Eva HOECK, München (DE)

(73) Assignee: Carl Zeiss Spectroscopy GmbH, Jean (DE)

(21) Appl. No.: 19/485,720

(22) PCT Filed: May 16, 2024
(86) PCT No.: PCT/EP2024/063457

§ 371 Date: Nov. 19, 2025

(30) Foreign Application Priority Data
May 25, 2023 (DE) 10 2023 113 704.2

(51) Int. Cl. G06T 7/00; G01N 21/25; H04N 23/21

(52) U.S. Cl. CPC G06T 7/0012; G01N 21/255; H04N 23/21; G01N 2201/0221; G06T 2207/30168; G06T 2207/30188

(57) ABSTRACT

For performing a condition analysis of a plant, an apparatus includes a near-field camera and a spectral analytical acquisition device. The near-field camera has a first field of view relative to a housing of the apparatus. The spectral analytical acquisition device has a second field of view relative to the housing, wherein the second field of view is completely contained within the first field of view.

### REFERENCE TO RELATED APPLICATIONS

This application claims the benefit of International Patent Application Serial Number PCT/EP2024/063457, filed on May 16, 2024; and German Patent Application Number 102023113704.2, filed on May 25, 2023; the disclosures of which are incorporated herein by reference.

### TECHNICAL FIELD

The invention relates to apparatus, evaluation devices or evaluation systems and methods usable for a condition analysis of a plant. The invention relates in particular to such apparatus, systems and methods which can support wavelength-resolved data acquisition, on the basis of which a condition of the plant, e.g., a supply of nutrients and/or water as well as pathological conditions, can be detected. The invention relates in particular to such apparatus, systems and methods which can be used with crop plants or cultivated plants.

### BACKGROUND

Analyzing the condition of plants is of great importance. It serves to ensure a sufficient supply of nutrients and/or to detect possible diseases.

Conventional methods for analyzing the condition of plants have traditionally relied on assessment by a human expert. Techniques that use measuring devices to objectively and quantitatively analyze plant conditions are becoming increasingly important. For example, such techniques can be used to detect an oversupply or undersupply of certain nutrients.

WO 2019/169434 A1, US 7 804 588 B2, US 11 320 307 B2 and DE 10 2018 103 509 disclose exemplary techniques for this. The sensor with the designation HarvestLab™3000, which is described in the product brochure “JOHN DEERE TECHNOLOGIES FOR PRECISION AGRICULTURE”, YY2314884GER_EN 01/23, comprises a near-infrared sensor.

Even when using modern (e.g. spectral analytical) measurement techniques for a condition analysis, data acquisition can require expert knowledge in the field. Data collection at an unfavorable location on the plant can lead to incorrect results of the condition analysis. An example of this is data acquisition of spectral analytical data at a point where foreign substances are present on a leaf of the plant. Data acquisition that relies solely on the knowledge and experience of a human expert to select a suitable location can result in problems of lack of objectivity of the assessment and/or lack of comparability. Automated data acquisition from a large number of locations on the same plant allows a subsequent selection to be performed, but has the disadvantage that large amounts of data have to be acquired, possibly transferred, and analyzed.

Thus, there is still a need for methods, apparatus and systems that offer machine-based support, optionally even further automation, in data acquisition, transmission and/or evaluation for analyzing the condition of plants.

### SUMMARY

The objective underlying the invention is to provide improved methods, apparatus and systems which can be used for analyzing the condition of plants. In particular, it is an objective underlying the invention to provide methods, apparatus and systems which provide improvements with regard to the acquisition, transmission and/or evaluation of spectral analytical information for analyzing a condition of plants. It is also an objective underlying the invention to provide such methods, apparatus and systems with which an acquisition, transmission and/or evaluation of spectral analytical information on useful plants or cultivated plants is guided, otherwise supported and/or simplified.

According to the invention, there are provided an apparatus, an evaluation device or an evaluation system and methods as defined by the independent claims. The dependent claims define preferred and advantageous embodiments.

According to one aspect, the invention relates to an apparatus for a condition analysis of a plant. The apparatus comprises a housing, a near-field camera, and a spectral analytical acquisition device. The near-field camera is configured to acquire at least one image of at least a part of the plant. The spectral analytical acquisition device is configured to acquire spectral analytical information of the plant. The near-field camera has a first field of view relative to the housing. The spectral analytical acquisition device has a second field of view relative to the housing. The second field of view is completely contained within the first field of view. The apparatus comprises at least one interface configured to output the at least one image and the spectral analytical information or data derived therefrom.

The apparatus provides various technical effects and advantages. The apparatus enables the acquisition of the at least one image that can be used to support data acquisition by the spectral analytical acquisition device or evaluation of the data acquired by the spectral analytical acquisition device. This can be done, for example, by user guidance during data acquisition by the spectral analytical acquisition device, wherein the user guidance depends on the at least one image acquired by the near-field camera. Alternatively or additionally, the image acquired by the near-field camera can be used to automatically control the spectral analytical acquisition device. With the second field of view being completely contained in the first field of view, it can be ensured that image information that can be captured with the near-field camera is available at all points for which data can be captured by the spectral analytical acquisition device. As a result, the usually more time-consuming acquisition and/or evaluation of the spectral analytical data can be made more efficient and less error-prone, using the at least one image acquired with the near-field camera. The at least one image acquired with the near-field camera allows an objective assessment and support of the data acquisition by the spectral analytical acquisition device. Alternatively or additionally, the at least one image acquired with the near-field camera allows an objective assessment and support of the data evaluation of the spectral analytical information. This reduces human error sources. By combining the near-field camera and the spectral analytical acquisition device in the apparatus (in particular in the same housing), a simple and reliable data acquisition for a condition analysis can be achieved.

The apparatus may be configured such that the at least one image can be used to determine a suitability assessment, for the acquisition or evaluation of the spectral analytical information, of the area of the plant represented in the at least one image.

Thereby, the apparatus makes it possible to use the at least one image acquired with the near-field camera to assess whether and/or at which positions on the plant (for example in which leaf areas) acquisition of the spectral analytical information makes sense, prior to performing the data acquisition. Data acquisition of spectral analytical information acquired at unsuitable positions of the plant can be avoided or reduced. Alternatively or additionally, the apparatus makes it possible to use the at least one image acquired with the near-field camera to assess, prior to data evaluation of the spectral analytical information, whether an evaluation of the spectral analytical information acquired at one or more specific plant positions (for example leaf areas) is meaningful. Alternatively or additionally, the apparatus enables the quality of the evaluation result to be assessed on the basis of the at least one image acquired with the near-field camera after data evaluation of the spectral analytical information. The at least one image acquired with the near-field camera is used for the assessment. This reduces sources of human error. The acquisition and/or evaluation of spectral analytical information acquired at unsuitable leaf locations, for example, can be avoided, which offers particular advantages in terms of processing time. Alternatively or additionally, after evaluation of the spectral analytical information, a quality assessment can be performed using the at least one image from the near-field camera in order to sort out possibly falsified evaluation results or to assign them a low weighting in relation to other evaluation results of the spectral analytical information.

In order to enable the determination of such a suitability assessment, the apparatus may comprise a controllable light source for illumination during the acquisition of the at least one image with the near-field camera. Alternatively or additionally, the apparatus may be configured to reduce ambient light influences, in particular to shield them, while the at least one image is acquired with the near-field camera.

This enables the acquisition of the at least one image under consistent conditions. This allows an objective assessment of the suitability, for the acquisition, analysis or subsequent evaluation of analysis results of the spectral analytical information, to be ascertained, for example, of the plant part (for example a leaf) shown in the at least one image as a whole or of different leaf parts or areas.

The spectral analytical acquisition device may be configured to perform the acquisition of the spectral analytical information based on the at least one image, for example based on the suitability assessment.

Thereby, acquisition of the spectral analytical information can be performed based on the at least one image acquired with the near-field camera. This can be done by providing user guidance during the acquisition of the spectral analytical information or by automatic control of the spectral analytical acquisition device. Data acquisition of spectral analytical information acquired at unsuitable positions on the plant can be avoided or reduced. Human error sources are reduced.

The apparatus can be configured to transmit the spectral analytical information or the data derived therefrom via the interface based on the at least one image, for example based on the suitability assessment, or to analyze it based on the at least one image, for example based on the suitability assessment.

Thereby, a transmission or analysis of the spectral analytical information or data derived therefrom can be performed based on the at least one image acquired with the near-field camera. An analysis or transmission of spectral analytical information acquired at unsuitable positions of the plant can be avoided or reduced. Human error sources are reduced.

The suitability assessment may comprise a suitability of the plant part (for example a leaf) represented in the at least one image for the acquisition and/or analysis of the spectral analytical information. The suitability assessment may be a binary value (suitable/unsuitable). The suitability assessment may be a value selected from a range of values that quantifies the suitability of the plant part (for example, a leaf) represented in the at least one image for the acquisition and/or evaluation of the spectral analytical information.

Thereby, the apparatus can be used to check the suitability of the plant part that is currently positioned against the apparatus for the acquisition of the spectral analytical information and/or its evaluation for the condition analysis.

The value may comprise a value from a continuous range of values. Thereby, it can continuously quantify the suitability with a value from an interval from a lower limit (for example 0%) to an upper limit (for example 100%).

This allows the suitability to be quantified.

The value may comprise a value from a discrete set of values, preferably from an ordinal scale of values. Thereby, it can indicate various, easily understandable different ratings.

Thereby, the suitability can be determined in such a way that it is also easy for a user to understand.

The suitability assessment may comprise a suitability of different positions or areas of the plant for a determination of nutrient concentrations. Alternatively or additionally, the suitability assessment may comprise a suitability of different positions or areas of the plant for a determination of a health status. For example, the suitability assessment may comprise a suitability of different positions or areas of the plant for determining a water content and/or pathological conditions. The determination can be based on the spectral analytical information.

Thereby, depending on which further analysis is to be carried out using the spectral analytical information, the positions or areas of the plant at which the spectral analytical information is to be acquired can be assessed automatically (in particular with processor support) using the at least one image acquired with the near-field camera in order to enable an automatic, spectral analytical determination of nutrient concentrations, health status, water content and/or pathological conditions that is as unbiased as possible.

The apparatus may be configured such that the spectral analytical acquisition device performs the acquisition of the spectral analytical information based on the suitability of different positions or areas. The apparatus may be configured to control the spectral analytical acquisition device based on the at least one image acquired by the near-field camera. The control may comprise an active adjustment of optical components to shift the second field of view relative to the housing.

Thereby, acquisition of the spectral analytical information can be carried out based on the at least one image acquired with the near-field camera. This can be done by automatically controlling the spectral analytical acquisition device. Data acquisition of spectral analytical information acquired at inappropriate positions of the plant can be avoided or reduced. Human error sources are reduced.

The apparatus may be configured to receive the suitability assessment via the interface.

This enables determination of the suitability assessment by at least one computing system (for example, a computer or server system) remotely from the apparatus. This allows the apparatus to be configured as a compact unit, while the suitability assessment can be determined remotely from the apparatus. To determine the suitability assessment, the apparatus may be configured to transmit the at least one image acquired by the near-field camera via the interface and then receive the suitability assessment.

The apparatus may be configured to determine the suitability assessment based on the at least one image acquired by the near-field camera.

This allows the suitability assessment to be determined locally in the apparatus. This can be particularly advantageous if appropriate computing resources are provided in the apparatus.

The apparatus may comprise a data processing circuit configured to process the at least one image acquired by the near-field camera. The data processing circuit may be configured to perform image segmentation of the at least one image to determine the suitability assessment. The data processing circuit may be configured to enable or automatically control of the spectral analytical acquisition device based on the image segmentation.

Thereby, the at least one image acquired by the near-field camera can be processed automatically.

The suitability assessment may define the suitability of several different positions or areas on a leaf of the plant for the acquisition and/or evaluation of the spectral analytical information.

Thereby, the at least one image acquired with the near-field camera can be used to quantitatively determine at which positions or areas of the leaf the acquisition and/or evaluation of the spectral analytical information is useful.

Alternatively or additionally, the at least one image acquired with the near-field camera can be used to assess an evaluation of the spectral analytical information as a function of the position or area of the leaf at which the spectral analytical information is or was acquired.

The suitability assessment may define a suitability pixel by pixel or area by area.

Thereby, the at least one image acquired with the near-field camera can be used to quantitatively determine at which locations or areas of the leaf the acquisition and/or evaluation of the spectral analytical information is useful.

The suitability assessment may comprise a numerical value for several pixels of the at least one image acquired with the near-field camera, which indicates whether reliable condition analysis results can be expected for a location defined by the pixel or an area defined by the pixel. The numerical value may be a binary value indicating whether or not the location or area is suitable for spectral analytical data acquisition for condition analysis. The numerical value may be selected from a range of values to further quantify suitability.

This can be used to determine which locations or areas are suitable for the acquisition of spectral analytical information. Alternatively or additionally, it may be determined which location or area is most suitable for acquiring the spectral analytical information.

The suitability assessment may indicate at which pixels of the at least one image acquired with the near-field camera on the imaged plant part foreign substances (for example liquids, dust or other particles or insects) are superimposed and/or at which pixels of the at least one image acquired with the near-field camera a degenerated state, such as necrosis, chlorosis, anthocyanosis (i.e. an accumulation of anthocyanins, which is noticeable for numerous plant species by a discoloration of leaves), disease or mechanical damage, is present.

This allows the spectral analytical information to be acquired and/or analyzed specifically at a location on the plant where the informative value of a condition analysis is not impaired by foreign substances or local degeneration.

The apparatus may comprise a device for reducing ambient light influences. The device for reducing ambient light influences may comprise an aperture provided on the housing. The aperture may at least partially protrude outwardly around a viewing window supported in the housing.

Thereby, the acquisition of the at least one image under consistent conditions is facilitated. This allows an objective, automatic assessment of the suitability of, for example, different leaf locations or areas for the acquisition, analysis or subsequent evaluation of spectral analytical information analysis results.

The near-field camera may comprise a camera and near-field optics to record a plant part positioned against the aperture.

This allows the at least one image to be captured under consistent conditions and in a field situation in which the apparatus is already positioned in contact with a possible sample location.

The apparatus may be configured to acquire the at least one image and the spectral analytical information while the apparatus (for example, the aperture) is positioned against a leaf of the plant.

This facilitates the acquisition of the at least one image under consistent conditions.

The apparatus may comprise a light source for providing illumination during acquisition of the at least one image with the near-field camera.

The acquisition of the at least one image under consistent conditions is facilitated thereby. This allows an objective, automatic assessment of the suitability of, for example, different leaf locations or areas for the acquisition, analysis or subsequent evaluation of spectral analytical information analysis results.

The apparatus may comprise illumination optics configured to illuminate at least the first field of view during the acquisition of the at least one first image. The illumination optics can be configured in such a way that light rays from a light source impinge on the sample as parallel rays and are picked up by the camera optics due to the sample reflection.

This achieves good illumination under consistently reproducible conditions. This facilitates the automatic evaluation of the at least one image.

The illumination optics may comprise at least one paraboloid reflector and/or at least one facetted reflector.

This enables consistent illumination with parallel light during the acquisition of the at least one image.

The illumination optics can be configured such that the light source acts as a point light source in the sample measurement plane.

This enables consistent illumination with parallel light during the acquisition of the at least one image.

Alternatively or additionally, the apparatus may be configured for calibration or referencing of the camera by means of a diffuse white/grey standard when the apparatus is initialized. This can happen once or recurrently. In the case of recurring white/grey standard measurements, intensity and/or spectral changes in the light source can be detected and compensated for.

This facilitates automatic evaluation of the at least one image to determine the suitability assessment.

The near-field camera may comprise at least one color channel or at least two color channels or three or more color channels. The near-field camera may comprise a camera with three color channels, for example an RGB camera.

This facilitates the automatic determination of the suitability assessment. For illustration, a color camera image (e.g. with RGB-color channels) can be advantageous, for example to assess the plant leaf stage or condition or to distinguish between color inhomogeneities and glare over the leaf surface.

The apparatus may be configured as a mobile device.

This allows it to be used in field operation on living plants.

The apparatus may be configured as a handheld device.

This facilitates use in field operation on living plants without necessarily having vehicle access to the plant.

The apparatus can be attached to a vehicle, in particular to an agricultural vehicle. Accordingly, according to an embodiment, a vehicle is provided that comprises an apparatus according to the invention mounted thereon.

This facilitates use in field operation on living plants using a vehicle.

The apparatus may be attached to a robot. Accordingly, according to an embodiment, a robot is provided comprising the apparatus according to the invention. The robot may be movably mounted, for example on a rail system or otherwise.

This facilitates its use, for example in greenhouses.

The apparatus can be attached to an aerial vehicle. Accordingly, according to an embodiment, an aerial vehicle is provided comprising the apparatus according to the invention.

This facilitates use in field operations on living plants using the aerial vehicle.

The vehicle, robot or aerial vehicle may comprise a receiver for receiving remote control signals and at least one actuator for moving the vehicle, robot or aerial vehicle in response to the remote control signals, in order to position the apparatus.

This enables control from a control position remote from the apparatus.

The vehicle, robot or aerial vehicle may comprise a controller configured to move the vehicle, robot or aerial vehicle autonomously or semi-autonomously to position the apparatus.

This achieves a higher degree of automation.

The vehicle, robot or aerial vehicle may comprise a gripper arm for gripping and positioning a plant part relative to the apparatus.

This allows plant parts that are more difficult to access to be correctly positioned relative to the apparatus.

According to a further aspect or embodiment, there is provided an evaluation device or evaluation system for a condition analysis of a plant, comprising: at least one evaluation device interface configured to receive at least one image of at least a part of the plant, and an evaluation circuit. The evaluation circuit is configured to determine, based on the at least one image, a suitability assessment of the area of the plant represented in the at least one image for the acquisition or evaluation of spectral analytical information.

The evaluation device or evaluation system provides various technical effects and advantages. The evaluation device or evaluation system uses the at least one image to support data acquisition by the spectral analytical acquisition device or evaluation of the data acquired by the spectral analytical acquisition device. Thus, the at least one image (which may be or which may comprise at least one near-field image) may be used to support the condition analysis based on the spectral analytical information.

The evaluation circuit may be configured to recognize, for determining the suitability assessment, one or more conditions selected from a group consisting of: presence of foreign substances, necrosis, chlorosis, anthocyanosis, degraded plant areas, pest infestation, misalignment of the area of the plant during image capture, crack or break in a part of the plant.

This makes it possible to determine which locations or areas are suitable for the acquisition or evaluation of spectral analytical information. Alternatively or additionally, it may be determined which location or area is most suitable for acquiring or evaluating the spectral analytical information.

The suitability assessment may comprise a suitability of the plant part (for example a leaf) represented in the at least one image for the acquisition and/or evaluation of the spectral analytical information. The suitability assessment may be a binary value (suitable/unsuitable). The suitability assessment may be a value selected from a range of values that quantifies the suitability of the plant part (for example, a leaf) represented in the at least one image for the acquisition and/or evaluation of the spectral analytical information.

Thereby, the suitability of the plant part represented in the at least one image for the acquisition of the spectral analytical information and/or its evaluation for condition analysis can be checked.

The suitability assessment may indicate, pixel by pixel or area by area, which locations or areas are suitable for the acquisition or evaluation of the spectral analytical information.

Thereby, the locations or areas of a leaf at which the acquisition and/or evaluation of the spectral analytical information is useful can be determined in a quantitative manner using the at least one image.

The suitability assessment may comprise a numerical value for several pixels of the at least one image acquired with the near-field camera, which indicates whether reliable condition analysis results are to be expected for a location or an area defined by the pixel. The numerical value may be a binary value indicating whether or not the location or area is suitable for spectral analytical data acquisition for condition analysis. The numerical value may be selected from a range of values to further quantify suitability.

Thereby, it can be determined which locations or areas are suitable for the acquisition of spectral analytical information. Alternatively or additionally, it can be determined which location or area is most suitable for acquiring the spectral analytical information.

The evaluation device or the evaluation system may be configured to take into account a number and/or geometric arrangement of several pixels relative to each other when determining the suitability assessment. For example, the evaluation device or the evaluation system may be configured to assess an area as suitable for spectral analytical acquisition only if at least two or more conditions are cumulatively fulfilled, such as: (a) none of the checked possible disturbing factors are present at the pixels; (b) the total number of such adjacent pixels satisfies a threshold comparison (for example, is greater than a threshold); (c) the shape and/or total area of the adjacent pixels satisfies one or more geometric criteria (for example, a size of more than a predetermined minimum dimension in one or two mutually orthogonal directions, a size that allows a circle with a predetermined minimum dimension to be inscribed, and/or an aspect ratio of the region defined by the pixels satisfies another threshold condition).

This can ensure that a region—for example, at least the second field of view—is free from possible disturbing factors in order to acquire the spectral analytical information. This enables more precise control, for example by defining maximum areas where certain disturbing factors must not be present.

The suitability assessment can indicate at which pixels of the at least one image acquired with the near-field camera on the imaged plant part foreign substances (for example liquids, dust or other particles or insects) are superimposed and/or at which pixels of the at least one image acquired with the near-field camera a degenerated state, such as necrosis, chlorosis, anthocyanosis or mechanical damage, is present.

This allows the spectral analytical information to be acquired and/or analyzed specifically at a location on the plant where the informative value of a condition analysis is not impaired by foreign substances or local degeneration.

The evaluation device or the evaluation system may comprise a human-machine interface. The human-machine interface may be configured to receive user input specifying one, more, or all of the following: plant stage, leaf stage, plant species, plant variety, at least one nutrient to be analyzed, at least one disease to be tested for.

This allows the evaluation device or the evaluation system to evaluate the at least one image based on the user input. In particular, this can prevent unnecessary, unwanted or unsuitable evaluations being carried out for the sample in question.

The evaluation circuit may be configured to perform the suitability assessment depending on the indication or indications contained in the user input.

Thereby, the evaluation device or the evaluation system can evaluate the at least one image based on the user input in such a way that those areas or locations (for example of a leaf) are determined at which a condition analysis specified by the user input is reliable on the basis of the spectral analytical information.

The at least one evaluation device interface may be configured to output the suitability assessment for use in the acquisition of spectral analytical information.

Thereby, the acquisition of the spectral analytical information may be performed by an apparatus separate from the evaluation device or evaluation system according to the suitability assessment.

The at least one evaluation device interface may be configured to transmit the suitability assessment to the apparatus according to an aspect or embodiment of the invention. The at least one evaluation device interface may be configured to receive the at least one image and the spectral analytical information from the apparatus.

Thereby, the evaluation device or the evaluation system is specifically adapted for interaction with the apparatus according to the invention.

The evaluation device or the evaluation system may be configured to control the at least one evaluation device interface or the human-machine interface to enable a user-defined selection of leaf areas to be analyzed.

This enables a user-defined selection of the leaf areas to be analyzed, which can be carried out at the evaluation device or the evaluation system or, under control thereof, at the apparatus according to the invention.

The evaluation circuit may be configured to carry out the suitability assessment without using spectral analytical information.

Thereby, the evaluation device or the evaluation system can determine, prior to performance of the more time-consuming acquisition of the spectral analytical information and on the basis of the at least one image, at which locations or areas of the plant the spectral analytical information is to be acquired in order to be able to reliably carry out the condition analysis.

The evaluation circuit may be configured to evaluate the at least one image using at least one trained machine learning model for determining the suitability assessment.

This allows the evaluation device or the evaluation system to perform the suitability assessment in a data-driven and objective manner. Annotated image data of plants or plant parts are used to train the machine learning model, with the annotations indicating which locations or areas are suitable for a condition analysis based on spectral analytical information. Expert knowledge is required to create the annotated training data, but not during inference of the trained machine learning model.

The trained machine learning model may be configured to receive pixel values of the at least one image as input.

This allows the at least one image to be processed in a simple manner by the evaluation device or the evaluation system. Optional preprocessing of the at least one image, for example by applying filters, edge detection methods or other preprocessing techniques, is possible.

The trained machine learning model may be configured to output the suitability assessment as an output.

The evaluation device interface may be configured to receive spectral analytical information of the plant or data derived from it.

This allows the evaluation device or evaluation system to not only determine the suitability assessment, but to perform the condition analysis also based on the spectral analytical information.

The evaluation device or the evaluation system can be configured to evaluate the spectral analytical information in order to perform the condition analysis of the plant. In doing so, the evaluation device or the evaluation system may be configured such that the spectral analytical information itself, its evaluation or an assessment of the evaluation result is performed based on the suitability assessment determined from the at least one image.

This allows the evaluation device or the evaluation system to not only determine the suitability assessment, but also to use it in the condition analysis based on the spectral analytical information.

The evaluation circuit may be configured to perform at least one nutrient analysis based on the spectral analytical information or the data derived therefrom.

Thereby, the evaluation device or the evaluation system can perform a condition analysis comprising a determination of at least one nutrient concentration. The evaluation device or the evaluation system may be configured to determine for one or more nutrients, based on the evaluation of the spectral analytical information, whether there is an oversupply or undersupply of the nutrient to the plant for the respective nutrient.

The evaluation device or the evaluation system may be structurally integrated into the housing of the apparatus. The evaluation device or the evaluation system may be separate from the apparatus with which the at least one image and the spectral analytical information are acquired.

According to a further aspect of the invention, there is provided a system comprising the apparatus according to an aspect or embodiment and the evaluation device or evaluation system according to an aspect or embodiment.

The effects achieved with the system correspond to the effects described with reference to the apparatus according to the invention and the evaluation device or evaluation system according to the invention.

According to a further aspect of the invention, a method for a condition analysis of a plant is provided, the method comprising: receiving at least one image of the plant and determining a suitability assessment of the plant for an acquisition or evaluation of spectral analytical information of the plant, wherein the suitability assessment is determined based on the at least one image, and wherein the acquisition and/or evaluation of the spectral analytical information of the plant is performed depending on the suitability assessment.

The method provides various technical effects and advantages. The at least one image is used to support a data acquisition and/or evaluation of the spectral analytical information. Thereby, the at least one image (which may be or comprise at least one near-field image) may be used to support the condition analysis based on the spectral analytical information.

The method may be performed automatically by the evaluation device, the evaluation system, the apparatus or the system according to an aspect or embodiment.

The method may further comprise outputting the suitability assessment via a human-machine interface for visualizing the suitability assessment.

Thereby, the acquisition of the spectral analytical information may be guided.

The method may comprise recognizing one or more conditions selected from a group consisting of: presence of foreign substances, necrosis, chlorosis, anthocyanosis, degraded plant areas, pest infestation, misalignment of the area of the plant during image acquisition, crack or break in a part of the plant.

Thereby, it is possible to determine which locations or areas are suitable for the acquisition or evaluation of spectral analytical information. Alternatively or additionally, it can be determined which location or area is most suitable for acquiring or evaluating the spectral analytical information.

In the method, the suitability assessment may indicate, on a pixel-by-pixel or area-by-area basis, which locations or areas are suitable for the acquisition or evaluation of the spectral analytical information.

Thereby, the at least one image can be used to quantitatively determine at which positions or areas of a leaf the acquisition and/or evaluation of the spectral analytical information is useful.

In the method, the suitability assessment for a plurality of pixels of the at least one image acquired by a near-field camera may comprise a numerical value indicating whether reliable condition analysis results can be expected for a position or area defined by the pixel. The numerical value may be a binary value indicating whether or not the location or area is suitable for spectral analytical data acquisition for condition analysis. The numerical value may be selected from a range of values to further quantify suitability.

Thereby, it can be determined which locations or areas are suitable for the acquisition of spectral analytical information. Alternatively or additionally, it can be determined which location or area is most suitable for acquiring the spectral analytical information.

In the method, the suitability assessment may indicate a suitability of the plant part (for example, a leaf) represented in the at least one image for the acquisition and/or evaluation of the spectral analytical information. The suitability assessment may be a binary value (suitable/unsuitable). The suitability assessment may be a value selected from a range of values that quantifies the suitability of the plant part (for example, a leaf) represented in the at least one image for the acquisition and/or evaluation of the spectral analytical information.

Thereby, the at least one image can be used to check the suitability, for the acquisition of the spectral analytical information and/or its evaluation for condition analysis, of the plant part currently positioned against the apparatus used for the acquisition of the at least one image.

The value may comprise a value from a continuous range of values. This can continuously quantify the suitability with a value from an interval from a lower limit (for example 0%) to an upper limit (for example 100%).

Thereby, the suitability can be quantified.

The value may comprise a value from a discrete set of values, preferably from an ordinal scale of values. This can indicate various, easily understandable different ratings.

Thereby, the suitability can be determined in such a way that it is also easy for a user to understand.

In the method, the suitability assessment may indicate at which pixels of the at least one image acquired with the near-field camera on the imaged plant part foreign substances (for example liquids, dust or other particles or insects) are superimposed and/or at which pixels of the at least one image acquired with the near-field camera a degenerated condition, such as necrosis, chlorosis, anthocyanosis or mechanical damage, is present.

This allows the spectral analytical information to be acquired and/or analyzed specifically at a location on the plant where the informative value of a condition analysis is not impaired by foreign substances or local degeneration.

The method may comprise controlling a human-machine interface to enable receiving a user input specifying one, more, or all of the following: plant stage, leaf stage, plant species, plant variety, at least one nutrient to be analyzed, at least one disease to be tested for.

Thus, based on the user input, the at least one image can be evaluated to determine the suitability assessment that is specifically adapted to the user-specified information.

In the method, the suitability assessment may be performed depending on the indication or indications contained in the user input.

Thus, depending on the user input, the at least one image can be evaluated to determine those areas or locations (for example of a leaf) where a condition analysis, based on the spectral analytical information and in accordance with the user input, is reliable.

The method may comprise outputting the suitability assessment for use in the acquisition of spectral analytical information.

Thereby, the acquisition of the spectral analytical information may be performed in accordance with the suitability assessment.

In the method, the suitability assessment may be transmitted to the apparatus according to an aspect or embodiment of the invention.

This allows the evaluation device or evaluation system to specifically interact with the apparatus according to the invention.

The method may comprise controlling an evaluation device interface or a human-machine interface to enable a user selection of leaf areas to be analyzed.

This enables a user-defined selection of the leaf areas to be analyzed, which can be performed at the evaluation device or the evaluation system or, under control thereof, at the apparatus according to the invention.

In the method, the suitability assessment can be performed without using spectral analytical information.

Thereby, even prior to the more time-consuming acquisition of the spectral analytical information, it can be determined on the basis of the at least one image at which locations or areas of the plant the spectral analytical information is to be acquired in order to be able to reliably perform the condition analysis.

The method may comprise an evaluation of the at least one image using at least one trained machine learning model to perform the suitability assessment.

This allows the suitability assessment to be data-driven and objective. Annotated image data of plants or plant parts are used to train the machine learning model, with the annotations respectively indicating which locations or areas are suitable for a condition analysis based on spectral analytical information. Expert knowledge is required to create the annotated training data, but not during inference with the trained machine learning model.

The trained machine learning model may be configured to receive pixel values of the at least one image as input.

This allows the at least one image to be processed in a simple manner by the evaluation device or the evaluation system. Optional preprocessing of the at least one image, for example by applying filters, edge detection methods or other preprocessing techniques, is possible.

The trained machine learning model may be configured to output the suitability assessment as an output.

The method may further comprise receiving spectral analytical information of the plant or data derived therefrom.

Thereby, not only the suitability assessment can be determined, but also the condition analysis can be performed based on the spectral analytical information.

The method may further comprise evaluating the spectral analytical information to perform the condition analysis of the plant. In this case, the spectral analytical information itself, its evaluation or an assessment of the evaluation result may be dependent on the suitability assessment determined from the at least one image.

As a result, not only can the suitability assessment be determined, but the condition analysis can also be performed based on the spectral analytical information.

The method may comprise performing at least one nutrient analysis based on the spectral analytical information or the data derived therefrom.

Thereby, a condition analysis comprising a determination of at least one nutrient concentration can be performed. For one or more nutrients, depending on the evaluation of the spectral analytical information, it can be determined whether the plant is oversupplied or undersupplied with the respective nutrient.

The method may comprise using a result of the condition analysis to adjust cultivation conditions.

Thereby, the result of the condition analysis can be used to improve, on the basis of the condition analysis, a supply of useful plants or cultivated plants.

The method may comprise acquiring the at least one image and/or the spectral analytical information by the apparatus according to an aspect or embodiment.

Thereby, the effects described with reference to the apparatus according to the invention may be achieved.

The method may comprise receiving the suitability assessment by the apparatus, wherein an acquisition of the spectral analytical information is performed depending on the suitability assessment.

As a result, the time-intensive acquisition of the spectral analytical information can be targeted using the suitability assessment. The risk of human misjudgment and/or acquisition of the spectral analytical information at an inappropriate location is reduced.

According to a further aspect of the invention, there is provided a method of cultivating plants comprising the method of performing a condition analysis, wherein at least one cultivation condition is set depending on a result of the condition analysis.

Thereby, the result of the condition analysis can be used to improve a supply of useful plants or cultivated plants based on the condition analysis.

According to a further aspect of the invention, there is provided a method of cultivating plants comprising a condition analysis using the apparatus, the evaluation device or the evaluation system according to an aspect or embodiment of the invention.

Thereby, the effects described in association with the apparatus according to the invention and the evaluation device or the evaluation system according to the invention are attained.

In the method, the at least one image and the spectral analytical information can be acquired by the apparatus, wherein the evaluation device or evaluation system uses the at least one image acquired with the near-field camera to support the condition analysis based on the spectroscopic information.

In the method, at least one cultivation condition can be set depending on a result of the condition analysis.

Thereby, the result of the condition analysis can be used to improve a supply of useful plants or cultivated plants based on the condition analysis.

According to a further aspect of the invention, machine-readable instruction code is provided which, when executed by a programmable computing unit, executes the method according to an aspect or embodiment of the invention.

According to a further aspect of the invention, there is provided a storage medium having machine-readable instruction code stored thereon which, when executed by a programmable computing unit, executes the method according to an aspect or embodiment of the invention.

The apparatus, methods, systems and system components according to embodiments may also be used in other fields of agricultural engineering, for example for studies on animals.

According to a further aspect, the invention relates to an apparatus for analyzing the condition of animals. The apparatus comprises a housing, a near-field camera and a spectral analytical acquisition device. The near-field camera is configured to acquire at least one image of at least part of the animals. The spectral analytical acquisition device is configured to acquire spectral analytical information of the animals. The near-field camera has a first field of view relative to the housing. The spectral analytical acquisition device has a second field of view relative to the housing. The second field of view is completely contained within the first field of view. The apparatus comprises at least one interface configured to output the at least one image and the spectral analytical information or data derived therefrom.

According to a further aspect or embodiment, an evaluation device or evaluation system for a condition analysis of animals is provided comprising: at least one evaluation device interface configured to receive at least one image of at least a portion of the animals, and an evaluation circuit. The evaluation circuit is configured to determine, based on the at least one image, a suitability assessment of the area of the animals represented in the at least one image for the acquisition or evaluation of spectral analytical information.

According to a further aspect of the invention, there is provided a system comprising the apparatus for analyzing the condition of animals according to an aspect or embodiment and the evaluation device or evaluation system according to an aspect or embodiment.

According to a further aspect of the invention, a method for a condition analysis of animals is provided, wherein the method comprises: receiving at least one image of the animals and determining a suitability assessment of the animals for an acquisition or evaluation of spectral analytical information of the animals, wherein the suitability assessment is determined based on the at least one image, and wherein the acquisition and/or evaluation of the spectral analytical information of the animals is performed based on the suitability assessment.

Further advantageous features of these apparatus, methods, evaluation devices and evaluation systems for application to animals correspond to the features and effects explained with reference to plant analysis.

The apparatus, methods, systems and system components according to embodiments of the invention achieve various effects. In particular, the acquisition and/or evaluation of spectral analytical information may be supported using an image or images acquired with a near-field camera.

The apparatus, methods, systems and system components can be used in various fields. These include, but are not limited to, the condition analysis of useful plants or cultivated plants in agricultural engineering.

### BRIEF DESCRIPTION OF THE DRAWINGS

Embodiments of the invention are described with reference to the drawings. In the drawings, similar or identical reference signs designate elements with a similar or identical configuration and/or function.

FIG. 1 is a block diagram of an apparatus according to an embodiment.

FIG. 2 shows a schematic representation of the apparatus.

FIG. 3 is a schematic representation of fields of view of a near-field camera and a spectral analytical acquisition device of the apparatus.

FIG. 4 is a schematic representation of fields of view of a near-field camera and a spectral analytical acquisition device of the apparatus.

FIG. 5 is a flowchart of a method according to an embodiment.

FIG. 6 is a schematic representation of a system according to an embodiment.

FIG. 7 is a diagram for illustrating the operation of the system according to an embodiment.

FIG. 8 is a block diagram of an evaluation device or an evaluation system according to an embodiment.

FIG. 9 is a block diagram of an apparatus according to another embodiment.

FIG. 10 is a schematic representation of a plant part for explaining operation of embodiments.

FIG. 11 is a schematic representation of a human-machine interface for explaining the operation of embodiments.

FIG. 12 is a flowchart of a method according to an embodiment.

FIG. 13 is a diagram for illustrating operation of the evaluation device or evaluation system according to an embodiment.

FIG. 14 shows an implementation of a suitability assessment using a support vector machine in the evaluation device or evaluation system according to an embodiment.

FIG. 15 shows an implementation of a suitability assessment using a machine learning model configured as a neural network in the evaluation device or the evaluation system according to an embodiment.

FIG. 16 is a flowchart of a method according to an embodiment.

FIG. 17 is a schematic representation of spectral analytical information for illustrating operation of the evaluation device or the evaluation system according to an embodiment.

FIG. 18 is a schematic representation of a result of a principal component analysis to illustrate operation of the evaluation device or the evaluation system according to an embodiment.

FIG. 19 is a flowchart of a method according to an embodiment.

FIG. 20 is a block diagram of an apparatus according to another embodiment.

FIG. 21 is a schematic representation of a near-field camera of the apparatus according to embodiments.

FIG. 22 shows a schematic representation of the apparatus when it is designed as a hand-held device.

FIG. 23 shows a schematic representation of an agricultural vehicle according to an embodiment.

FIG. 24 shows a schematic representation of a robot according to an embodiment.

FIG. 25 shows a schematic representation of an aerial vehicle according to an embodiment.

FIG. 26 is a flowchart of a method according to an embodiment.

### DETAILED DESCRIPTION OF EMBODIMENTS

Embodiments of the invention are described with reference to the drawings. In the drawings, similar or identical reference signs designate elements with a similar or identical configuration and/or function.

While embodiments are described in association with a nutrient analysis or other condition analysis of a useful plant or cultivated plant, the embodiments are not limited thereto.

The features of the embodiments may be combined with each other, unless this is expressly excluded in the following description.

Apparatus, evaluation devices, evaluation systems and methods according to embodiments of the invention are configured to use at least one image acquired with a near-field camera, which shows at least part of a plant, to support an acquisition, evaluation or assessment of spectral analytical information. This can be done in different ways, for example by
using the at least one image acquired by the near-field camera to control a human-machine interface to indicate which locations or areas of a leaf, for example, are suitable for an acquisition of spectral analytical information,
using the at least one image acquired by the near-field camera to automatically control a spectral analytical acquisition device,
using the at least one image acquired by the near-field camera to determine whether acquired spectral analytical information is to be transmitted or evaluated to perform the condition analysis, and/or
using the at least one image acquired by the near-field camera to subsequently assess, for example weight, a condition analysis performed using the spectral analytical information.

The term spectral analytical information, as used herein, includes an optical measurand acquired for several (two or more than two) wavelengths at a same spatial measurement area of the plant. The measurand may comprise, for example, a reflectivity, intensity of reflected light, intensity of scattered light, intensity of Raman scattered light, intensity of fluorescent light, intensity of phosphorescent light or other optical measurand acquired for the several wavelengths, which may also be determined, for example, in a transmission set-up (for example, by acquisition of transmitted intensity as a function of wavelength) or a transflectance set-up.

The term spectral analytical acquisition device, as used herein, includes a device capable of and configured for acquiring an optical measurand for several (two or more than two) wavelengths at the same measurement area of the plant. The spectral analytical acquisition device may comprise a spectrometer. However, this is not necessarily required. For example, as an alternative or in addition to using a spectrometer, the spectral analytical acquisition device may be configured to acquire the optical measurand for the several wavelengths in a time-sequential manner, for example by actively irradiating different wavelengths and determining in each case the intensity of the scattered or reflected light detected.

The term near-field camera, as used here, includes in particular an image acquisition device with near-field optics that allows the sample (for example a leaf of the plant) to be positioned in proximity to the near-field camera (for example in direct contact with an aperture of an apparatus in whose housing the near-field camera is installed).

The term near-field image, as used herein, refers to an image captured by the near-field camera.

The term apparatus for a condition analysis of a plant, as used herein, refers to an apparatus usable for a condition analysis of a plant. The apparatus is specifically configured to acquire at least a part and advantageously all of the acquisition data (images and spectral analytical information) required for the condition analysis. It is possible, but not absolutely necessary, that the evaluation for the condition analysis is also performed by the apparatus itself. Rather, the term apparatus for a condition analysis of a plant also includes such apparatus that allows the acquisition of at least one image with a near-field camera and of spectral analytical information to be performed on an object (in particular a leaf of a plant or another plant part).

The term plant, as used herein, also includes eukaryotes, in particular algae.

According to the invention, techniques are provided which allow the condition analysis of a plant to be supported based on at least one image acquired with a near-field camera. In particular, a near-field camera comprising a near-field optical system and a spectral analytical acquisition device (for example a spectrometer) may be installed in a housing of an apparatus for this purpose. The at least one near-field image captured by the near-field camera is automatically analyzed. This can be done either locally in the apparatus or in a separate computing system.

According to the invention, an apparatus thus comprises both a near-field camera (which may comprise, in particular, a camera and a near-field lens) and a spectral analytical acquisition device. The near-field camera has a field of view that can be approximately equal to or larger than a field of view of the spectral analytical acquisition device. Thus, the at least one image acquired by the near-field camera covers at least the area of the object to be measured (in particular a plant part, for example a leaf) that is used for acquiring the spectral analytical information. The apparatus may first capture the at least one image of the object to be measured, using the near-field camera. The at least one image can be used to perform an automated evaluation of the object to be measured for a property intended for a condition analysis (such as absence of foreign substances, correct leaf side, correct positioning, absence of necrosis, absence of chlorosis, absence of anthocyanosis). This automated evaluation can be carried out by the apparatus itself or remotely from the apparatus by an evaluation device or an evaluation system that is in communication with the apparatus. The result of the evaluation of the at least one image can be used in a condition analysis based on spectral analytical information.

By analyzing the at least one image acquired with the near-field camera, it is possible, for example, to automatically detect locations and/or areas of a plant that are unsuitable for a condition analysis based on spectral analytical information. Root-causes for this can be, for example, foreign substances present on the plant, mechanical defects of the plant, diseases, necrosis, chlorosis or anthocyanosis. If such situations that impair the reliability of a condition analysis based on the spectral analytical information are detected, at least one automatic action can be taken, for example one or several of the following actions:
controlling a human-machine interface to indicate that the reliability of the condition analysis based on the spectral analytical information may be impaired;
automatically controlling the spectral analytical acquisition device to prevent acquisition of the spectral analytical information at locations where the reliability of the condition analysis based on the spectral analytical information may be impaired;
preventing further analysis of the spectral analytical information when the reliability of the condition analysis based on the spectral analytical information may be impaired; and/or
assessing a condition analysis, for example by low weighting of the analysis result, when the condition analysis has been performed based on spectral analytical information, if the reliability of the condition analysis based on the spectral analytical information may be impaired.

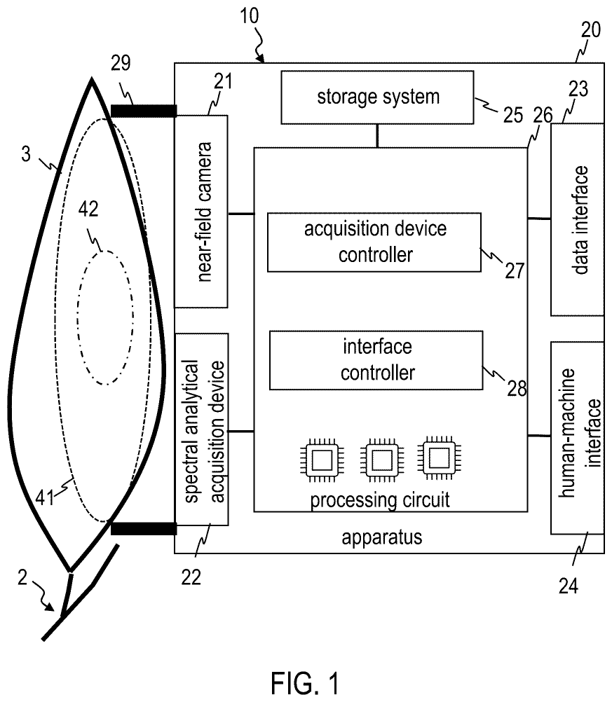

FIG. 1 shows an apparatus 10 for a condition analysis of a plant 2. The apparatus comprises a housing 20. The apparatus 10 comprises a near-field camera 21 arranged on or in the housing 20. The apparatus 10 comprises a spectral analytical acquisition device 22 arranged on or in the housing 20.

The near-field camera 21 is configured to acquire at least one image of at least a part (for example a leaf 3) of the plant 2. The spectral analytical acquisition device 22 is configured to acquire spectral analytical information of the plant. The near-field camera 21 has a first field of view 41 relative to the housing 20. The spectral analytical acquisition device 22 has a second field of view 42 relative to the housing. The second field of view 42 is completely contained within the first field of view 41. For illustration, the second field of view 42 and the first field of view 41 may be substantially identical. However, the second field of view 42 may also be smaller than the first field of view 41, in which case the second field of view 42 is contained within the first field of view.

The apparatus comprises at least one interface 23, 24 configured to output the at least one image and the spectral analytical information. The at least one interface 23, 24 may also be configured to output data derived from the at least one image and/or the spectral analytical information.

The at least one interface may comprise a data interface 23. The data interface 23 may comprise a wireless communication interface. The apparatus 10 may be configured to output the at least one image acquired with the near-field camera 21 (which is also referred to herein as a near-field image) or data derived therefrom via the data interface 23.

The apparatus 10 may be configured to receive an evaluation result determined from the at least one near-field image via the data interface. The evaluation result may comprise a suitability assessment indicating whether the plant section (for example leaf section) surrounded by an aperture 29 of the apparatus is suitable for data acquisition of the spectral analytical information and/or whether a condition analysis of spectral analytical information acquired in this plant section can be expected to yield reliable results.

The evaluation result of the at least one near-field image can be used by the apparatus 10 in different ways to influence the acquisition, assessment or subsequent evaluation of the spectral analytical information. In this way, the apparatus 10 can, e.g., support the acquisition and/or evaluation of the spectral analytical information in order to allow a reliable condition analysis.

The apparatus 10 may be configured to control the spectral analytical acquisition device 22 based on the evaluation result of the at least one near-field image. For example, acquisition of the spectral analytical information can be prevented or made dependent on a dedicated user input if the evaluation result of the at least one near-field image indicates that the plant section abutting the apparatus 10 is unsuitable for the acquisition and/or evaluation of the spectral analytical information. The spectral analytical acquisition device 22 can alternatively or additionally be controlled in such a way that the acquisition of the spectral analytical information is performed at a measurement spot in the first field of view 41 at which, based on the evaluation result of the at least one near-field image, such an acquisition is useful with regard to the condition analysis. The control of the spectral analytical acquisition device 22 may be performed by an acquisition device controller 27 of a processing circuit 26 of the apparatus 10. The control of a human-machine interface 24 to allow a dedicated user confirmation based on the analysis result of the at least one near-field image may be performed by an interface controller 28 of the processing circuit 26 of the apparatus 10.

The apparatus 10 may be configured to control the human-machine interface 24 of the apparatus based on the evaluation result of the at least one near-field image. For example, the human-machine interface 24 can be controlled to indicate visually, acoustically and/or in a tactile manner whether an acquisition of the spectral analytical information on the plant section currently abutting on the apparatus 10 is useful. The human-machine interface 24 may comprise a display device that can be controlled by the interface controller 28 such that, with respect to the plant section currently in contact with the apparatus 10, it is respectively indicated (for example in the form of overlay(s) on the near-field image) which measurement spots are suitable for the acquisition of the spectral analytical information.

The apparatus 10 may alternatively or additionally be configured to transmit the spectral analytical information via the data interface 23 for performing the condition analysis. The apparatus 10 may use the evaluation result of the at least one near-field image to selectively perform the transmission of the spectral analytical information only if it is determined, based on the evaluation result of the at least one near-field image, that the spectral analytical information is determined at a measurement spot in the first field of view 41 that is suitable for the condition analysis.

The apparatus 10 thus provides a device that allows a check to be performed whether an acquisition and/or evaluation of spectral analytical information in the plant section (for example leaf section) abutting on the apparatus 10 can be expected to provide reliable results of a condition analysis that is based on the spectral analytical information.

To perform the various control and processing functions, the apparatus 10 comprises a processing circuit 26. The processing circuit 26 may comprise one or several integrated circuits to perform control of the data interface 23, the human-machine interface, the spectral analytical acquisition device 22, and/or the near-field camera 21. The one or more integrated circuits may comprise, e.g., any one or any combination of the following circuits or circuit components: an application specific integrated circuit (ASIC), a field programmable gate array (FPGA), a processor (for example, a GPU (“graphic processor unit”) or TPU (“tensor processor unit”)), a controller, one or several quantum gates, a quantum information processing circuit, other integrated circuits.

The apparatus 10 may comprise a storage system 25. The storage system 25 may store machine-readable instruction code that, when executed by the processing circuit 26, causes execution of the functions and steps disclosed herein. If the evaluation of the at least one near-field image is performed by the processing circuit 26, i.e. locally in the apparatus 10, the storage system 25 may also store parameters that allow the at least one near-field image to be evaluated as to whether the imaged plant part is suitable for an acquisition and/or evaluation of the spectral analytical information. The parameters stored in the storage system 25 for evaluating the at least one near-field image may comprise, for example, one or several of the following parameters: Filter parameters; definition of decision boundaries of a support vector model; parameters of a trained machine learning model. Further examples are provided in more detail with reference to the evaluation device or the evaluation system.

The condition analysis based on the spectral analytical information can be performed locally in the apparatus 10 or, advantageously, in a computer system remote from the apparatus 10. The condition analysis, which may for example include the determination of nutrient concentrations based on the spectral analytical information, may be performed using techniques known to the skilled person, such as those described in WO 2019/169434 A1, US 7 804 588 B2 and US 11 320 307 B2. The condition analysis can be carried out using principal component techniques, partial least squares (PLS) regression techniques or other techniques that allow a conclusion to be drawn as to whether a nutrient concentration is within or outside a target range for at least one and advantageously several nutrients.

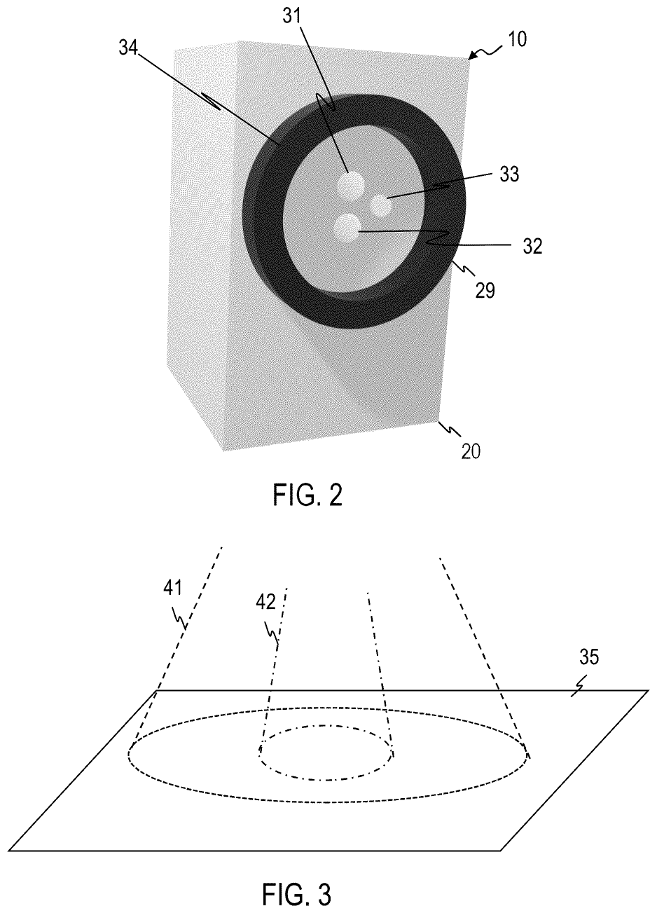

FIG. 2 shows a schematic perspective view of the apparatus 10. Various geometric shapes can be used for the housing of the apparatus 10 and the aperture 29. Advantageously, the aperture 29 defines a continuous contact surface 34 for direct contact with the plant section (e.g., a leaf). The aperture 29 reduces extraneous light influences.

A near-field optics 31 of the near-field camera 21 is arranged such that the near-field optics 31 images reflected and/or scattered light within the field of view surrounded by the contact surface 34 onto a detector surface (for example a light-sensitive semiconductor chip, for example a CMOS or CCD chip) of a camera. A light source is arranged to actively illuminate the field of view of the near-field camera surrounded by the contact surface 34. Illumination optics 33 can be configured to provide illumination with parallel light, for example.

The spectral analytical acquisition device 22 may comprise optical components 32 for directing a probe beam onto the plant section abutting on the abutment surface 34 within the field of view surrounded by the abutment surface 34 and acquiring the reflected or scattered light of the sample beam.

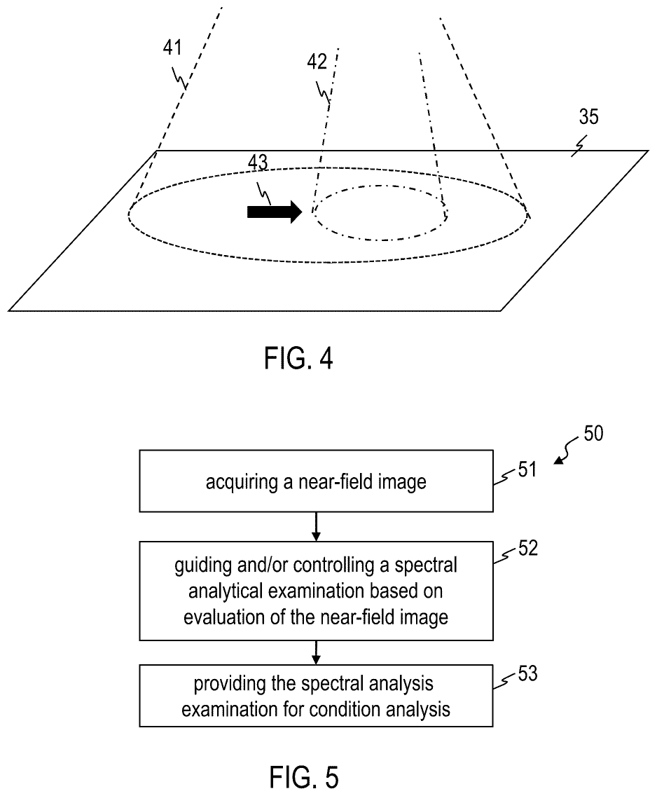

FIG. 3 and FIG. 4 show the first field of view 41 of the near-field camera 21 and the second field of view 42 of the spectral analytical acquisition device 22. The second field of view 42 may be substantially identical to the first field of view 41 or may be smaller than the first field of view 41. In either case, the first field of view 41 of the near-field camera 21 includes the second field of view 42 of the spectral analytical acquisition device 22.

The apparatus 10 may be configured to change a position and/or size of the second field of view 42 in an object plane 35 (which may be defined by the abutment surface 34). For example, a measurement position of the spectral analytical acquisition device can be displaced by a displacement 43. Advantageously, the position and/or size of that measurement position at which the spectral analytical information is acquired can then be based on the evaluation of the at least one near-field image. The processing device 26 can control the spectral analytical acquisition device 22 based on the evaluation of the at least one near-field image, optionally also based on a user input that is dependent on the evaluation of the near-field image. Thereby, spectral analytical information can be acquired in an area within the plant section in contact with the abutment surface 34, with the area being determined, based on the evaluation of the near-field image, to be suitable for the acquisition of the spectral analytical information for the purpose of the condition analysis.

The position of the second field of view 42 relative to the first field of view 41 can be determined and known during field operation. For example, a calibration can be performed at the factory and the corresponding position information can be stored in non-volatile memory in the apparatus 10. Alternatively or additionally, the calibration can, for example, also be carried out repeatedly after commissioning of the apparatus 10 to determine the position information relating to the position of the second field of view 42 relative to the first field of view 41. This location information may be used by the apparatus 10, for example, to acquire the spectral analytical information at a location determined to be suitable based on the suitability assessment referenced to the near field image.

Alternatively or additionally, the position information may also be used to visualize the position of the second field of view 42 relative to the first field of view 41. For example, the position may be displayed as superimposed information on the near-field image to visualize a measurement spot of the spectral analytical acquisition device. For this purpose, the human-machine interface 24 can be controlled based on the position information.

The determination of the position of the second field of view 42 relative to the first field of view 41 can be performed in various ways. Corresponding techniques are known to the person skilled in the art. For example, the position can be determined based on any of the following techniques:
Coupling light into a light path of the spectral analytical acquisition device to illuminate a sample (which may be, for example, a dedicated calibration sample) and acquiring the position of the spot (which corresponds to the measurement spot of the spectral analytical acquisition device) in the near-field image.
Positioning an optical fiber in front of the viewing window of the apparatus 10 and displacing the light emitting end of the optical fiber in two dimensions to scan the area of the viewing window. Based on the respective intensities acquired by the spectral analytical acquisition device, the position of the measurement spot of the spectral analytical acquisition device relative to the first field of view 41 can be determined.

For each pixel in the near-field image (first field of view 41), both techniques quantify how much information from this pixel will arrive at the spectral analytical acquisition device. This calibration information can be used in the acquisition and/or evaluation of the spectral analytical information, for example for visualization and/or evaluation of the assessment results. While with reference to FIG. 2, FIG. 3 and FIG. 4 an embodiment of the apparatus 10 with an aperture 29 has been described, other devices for reducing ambient light influences may alternatively or additionally be provided. For example, modulation techniques may be used in which the active illumination is temporally modulated and several near-field images are acquired during the temporal modulation. The several near-field images can be computationally processed in combination with information about the temporal modulation of the illumination to computationally suppress ambient light influences.

FIG. 5 shows a flowchart of a method 50. The method 50 may be automatically executed by the apparatus 10.

At step 51, a near-field image is acquired. At least one near-field image of a plant section abutting directly on the apparatus 10 (for example on the abutment surface 34 of the aperture 29) may be acquired.

At step 52, a spectral analytical examination of the plant section is guided and/or controlled and/or otherwise supported based on an evaluation of the at least one near-field image. This can be done in various ways, as already explained. In steps 51 and 52, the apparatus 10 can remain in contact with the plant section. A position of the apparatus 10 relative to the plant section in contact with it can remain essentially unaltered. Step 52 may comprise the acquisition of spectral analytical information based on the evaluation of the at least one near-field image. The acquisition of the spectral analytical information may be performed selectively based on the result of the evaluation of the at least one near-field image. The spectral analytical information may also be acquired independently of the result of the assessment of the at least one near-field image, with the result of the assessment of the at least one near-field image being used to determine whether the spectral analytical information can be used for the condition analysis or how the spectral analytical information is to be assessed (for example weighted) in the condition analysis. The assessment or weighting may also comprise a complete rejection or exclusion of spectral analytical information.

At step 53, a result of the spectral analytical examination (for example, an acquired spectrum or wavelengths of local maxima (peaks) of the acquired spectrum) is provided for use in the condition analysis. Step 53 may include performing the condition analysis based on the acquired spectral analytical information acquired during the spectral analytical examination. Alternatively or additionally, step 53 may comprise transmitting the spectral analytical information to a computer system remote from the apparatus 10. The computer system remote from the apparatus 10 may be configured as an evaluation device or evaluation system according to embodiments of the invention to perform the condition analysis based on the spectral analytical information.

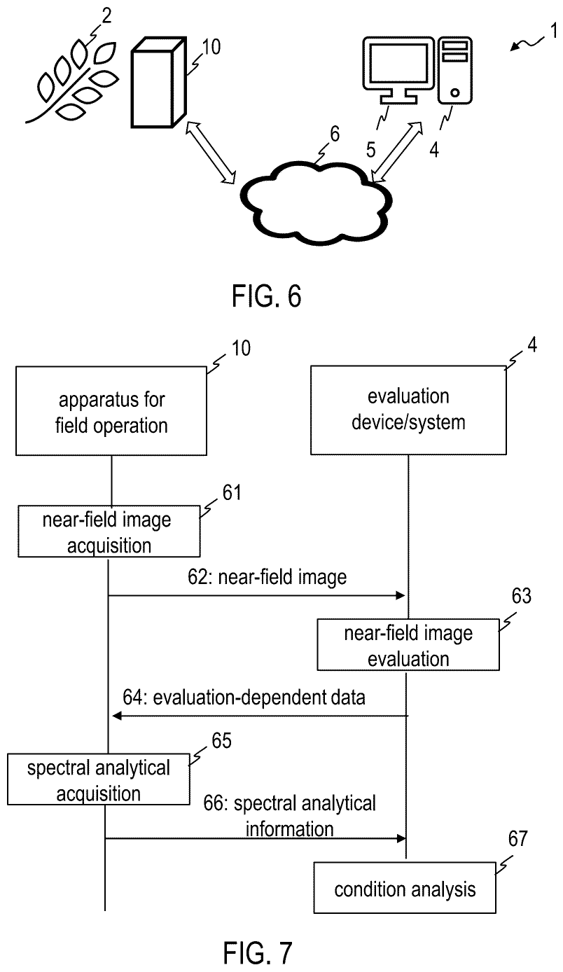

FIG. 6 is a schematic block diagram of a system 1 according to an embodiment. The system 1 comprises an apparatus 10 according to an embodiment. The system 1 comprises a computer system 4 separate from the apparatus 10. The computer system 4 may comprise a mobile communication terminal (for example a cell phone with a display device) and/or one or several computers or servers, which may be positioned remotely from the apparatus 10. A display device 5 can be coupled to the computer system 4. The computer system 4 may be at least selectively in communication with the apparatus 10. For this purpose, data can be transmitted between the apparatus 10 and the computer system 4 via a communication system comprising a wide area network 6, a wireless or wired point-to-point connection, a local area network and/or a wireless communication system. The data transmission between the apparatus 10 and the computer system 4 can be configured such that data can respectively be transmitted from the apparatus 10 to the computer system 4. Advantageously, bi-directional data transmission is provided, in which, for example, a result of an evaluation of a near-field image and/or a result of the condition analysis based on the spectral analytical information is transmitted from the computer system 4 to the apparatus 10. The apparatus 10 is positioned near the plant 2 to be examined during field operation.

In an embodiment, the system 1 comprises a communication terminal which is different from the apparatus 10 and is at least selectively in communication with the latter. The communication terminal may be configured to perform the functions of the evaluation device disclosed herein. The communication terminal may be configured to transmit acquired data (for example, near-field images and/or spectral analytical information) and/or information derived therefrom to a storage system remote from the apparatus 10.

In a further embodiment, the communication terminal may be configured to transmit the data acquired by the apparatus 10 (for example, near-field images and/or spectral analytical information) to a computer or server that performs the functions of the evaluation device or evaluation system disclosed herein. The communication terminal may receive the evaluation results (for example, the suitability assessment) from the evaluation device or evaluation system. The evaluation results can be used for the control of the apparatus 10.

FIG. 7 is a signal diagram for illustrating operation of the apparatus 10 and the evaluation device or evaluation system 4 according to an embodiment.

The apparatus 10 performs a near-field image acquisition 61 to acquire at least one near-field image of a plant section in contact with the apparatus 10. The near-field image 62 or data derived therefrom may be transmitted to the evaluation device or the evaluation system 4.

The evaluation device or the evaluation system 4 performs a near-field image evaluation 63 of the at least one near-field image. The near-field image evaluation 63 may comprise image analysis techniques known in the art. For example, one or several of the following techniques may be used: filtering (e.g., using edge enhancing filters); segmentation (e.g., based on color information and/or intensity information); processing using a trained machine learning model (as will be explained in more detail below). The result of the near-field image evaluation 63 may indicate whether the plant section abutting on the apparatus 10 is sufficiently intact and free of foreign substances to perform a condition analysis based on spectral analytical information detected on this plant section. The evaluation result may thus comprise a suitability assessment represented by a binary value (suitable/unsuitable) for the entire plant section represented in the at least one near-field image. The evaluation result may also indicate which areas and/or locations on the plant section are sufficiently intact and free of foreign substances to perform a condition analysis based on spectral analytical information acquired on that plant section.

The apparatus 10 may receive data 64 based on the evaluation result from the evaluation device or evaluation system 4. The data 64 may comprise a binary value (suitable/unsuitable), which indicates whether the plant section represented in the at least one near-field image is suitable for the acquisition and/or analysis of spectral analytical information for the respective condition analysis.

The apparatus 10 may perform an acquisition 65 of the spectral analytical information based on the data 64. During the time interval in which the near-field image acquisition 61 and the acquisition 65 of the spectral analytical information is performed, the apparatus 10 remains positioned in a substantially consistent position relative to the plant 2. The acquisition 65 may depend on the data 64 in various ways, as explained above.

The apparatus 10 may transmit the spectral analytical information 66 or data based thereon (for example, a location of local maxima of an acquired spectrum) to the evaluation device or evaluation system 4. The evaluation device or the evaluation system 4 can then perform the condition analysis based on the spectral analytical information 66 or data based on it. A result of the condition analysis can optionally be transmitted to the apparatus 10 and output at the human-machine interface of the apparatus 10.

Various modifications are possible. For example, the near-field image evaluation 63 may also be performed locally in the apparatus 10. The result of the near-field image evaluation may then optionally be transmitted to the evaluation device or the evaluation system 4 and used by the evaluation device or the evaluation system 4 in the condition analysis, which is performed on the basis of the spectral analytical information 66. In a further embodiment, the condition analysis 67 may also be performed locally in the apparatus 10. Further embodiments are possible in which the processing of the at least one near-field image and/or the spectral analytical information may be distributed in different ways between the apparatus and one or several optional further computing devices.

For example, it is possible for a near-field image captured by the apparatus 10 to be transmitted to a first computing device (for example, a communication terminal such as a cell phone) for processing. A local interface such as USB or Bluetooth can be used for this purpose. The first computing device can send the near-field image to a second computing device (for example, one or several computers or servers, which can also be arranged in a cloud architecture) via a WLAN interface or cellular interface. The second computing device can evaluate the near-field image to determine its suitability for spectral analysis data acquisition. The suitability assessment can be transmitted to the first computing device and used by the first computing device or the apparatus 10 to acquire the spectral analytical information, for example in the form of generating a recommended action. If the at least one plant part is suitable, the apparatus 10 may perform an acquisition of the spectral analytical information. The spectral analytical information may preferably be evaluated by the first computing device, optionally also by the second computing device. If the first computing device performs the evaluation of the spectral analytical information, the evaluation result of the spectral analytical information can be sent to the second computing device for archiving.

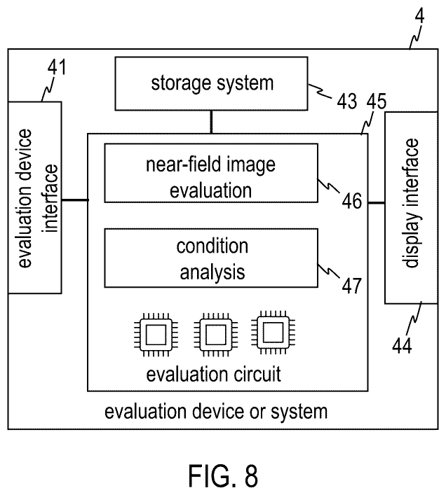

FIG. 8 is a schematic block diagram representation of an evaluation device 4. While the evaluation device 4 is shown as implemented in a unit, the various functions and components can also be implemented in a distributed evaluation system, for example in a server system or computer system.

The evaluation device 4 comprises an evaluation device interface 41 configured to receive the at least one near-field image and the spectral analytical information from the apparatus 10. The evaluation device interface 41 may also be configured to output an evaluation result of the at least one near-field image (in particular a suitability assessment for the acquisition of the spectral analytical information) and/or a result of the condition analysis based on the spectral analytical information.

The evaluation device 4 comprises at least one display interface 44. Via the display interface 44, the evaluation device 4 may control the display device 5 such that an evaluation result of the at least one near-field image and/or the result of the condition analysis is output. Alternatively or additionally, the display interface 44 may be used to control the display device 5 such that various user-defined inputs are allowed, such as an input of the plant variety or plant species, an input of the condition analysis to be performed, etc.

The evaluation device 4 comprises an evaluation circuit 45. The evaluation circuit 45 may be configured to perform the near-field image evaluation 46 to determine whether the plant section positioned against the apparatus 10 is suitable for the acquisition and/or evaluation of the spectral analytical information. The evaluation circuit 45 may be configured to perform a condition analysis 46 based on the spectral analytical information.

The evaluation device 4 may be configured to determine an evaluation result of the near-field image and transmit it to the apparatus 10 via the evaluation device interface 41. The evaluation result may comprise a suitability assessment indicating whether the plant section (e.g., leaf section) positioned on the apparatus 10 is suitable for data acquisition of the spectral analytical information and/or whether a condition analysis of spectral analytical information acquired in this plant section can be expected to produce reliable results. As explained above, the suitability assessment can be a single (e.g. binary) value that refers to the plant section as a whole. Other discrete ranges of values are possible, for example a suitability assessment for the plant section that expresses suitability in one of three possible values (for example in the manner of a traffic light system) or four possible values (for example “faultless”, “still good”, “less good”, and “unsuitable”) or more than four possible values. The suitability assessment can also be selected from continuous value ranges (for example from 0% to 100%), as already explained.

In each case, the suitability assessment may indicate, with respect to the first field of view 41, which areas and/or positions of the plant section in contact with the apparatus 10 are suitable for the acquisition of the spectral analytical information.

The evaluation device 4 can thus support a verification that is useful to determine whether an acquisition and/or evaluation of spectral analytical information in the plant section (for example, leaf section) positioned against the apparatus 10 can be expected to provide reliable results of a condition analysis based on the spectral analytical information.

The condition analysis 47 may include, for example, determining nutrient concentrations based on the spectral analytical information. The condition analysis 47 may be performed using techniques known to the skilled person, such as those described in WO 2019/169434 A1, US 7 804 588 B2 and US 11 320 307 B2. The condition analysis 47 may be performed using principal component techniques, PLS techniques or other techniques that allow a conclusion to be drawn as to whether a nutrient concentration is within a target range or outside a target range for at least one and advantageously several nutrients. The condition analysis 47 may also quantitatively determine the nutrient concentration for one or several nutrients. This value can then be used, for example, to check whether the nutrient concentration is within the target range (normal supply) or outside the target range (undersupply or oversupply).

To perform the various processing functions, the evaluation device 4 comprises the evaluation circuit 45. The evaluation circuit 45 may comprise one or several integrated circuits to perform the near-field image evaluation 46 and/or the condition analysis 47. The one or more integrated circuits may comprise, for example, any one or any combination of the following circuits or circuit components: an application specific integrated circuit (ASIC), a field programmable gate array (FPGA), a processor, a GPU, a TPU, a controller, one or several quantum gates, a quantum information processing circuit, other integrated circuits.

The evaluation device 4 may comprise a storage system 43. The storage system 43 may store machine-readable instruction code which, when executed by the evaluation circuit 45, causes execution of the functions and steps disclosed herein. When the evaluation of the at least one near-field image is performed by the evaluation circuit 45, the storage system 43 may store parameters that allow an evaluation of the at least one near-field image as to whether the imaged plant part is suitable for an acquisition and/or evaluation of the spectral analytical information. The parameters stored in the storage system 43 for evaluating the at least one near-field image may comprise, for example, one or several of the following parameters: filter parameters; definition of decision boundaries of a support vector model; parameters of a trained machine learning model. Further examples are described in more detail with reference to the evaluation device or the evaluation system.

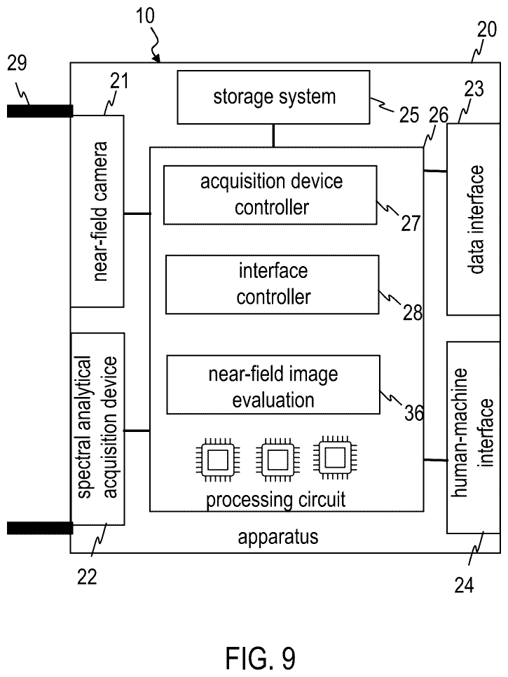

FIG. 9 shows a modification of the apparatus 10. The apparatus 10 is configured such that the near-field image evaluation can be performed locally in the apparatus 10. The processing circuit 26 of the apparatus 10 is configured to carry out the near-field image evaluation 36. The near-field image evaluation 36 can be realized as already explained above. In particular, the near-field image evaluation 36 may use automated image processing techniques (for example, the use of one or several trained machine learning models) to determine whether the plant section abutting on the apparatus 10 and accessible to the spectral analytical acquisition device is suitable for the acquisition of the spectral analytical information.

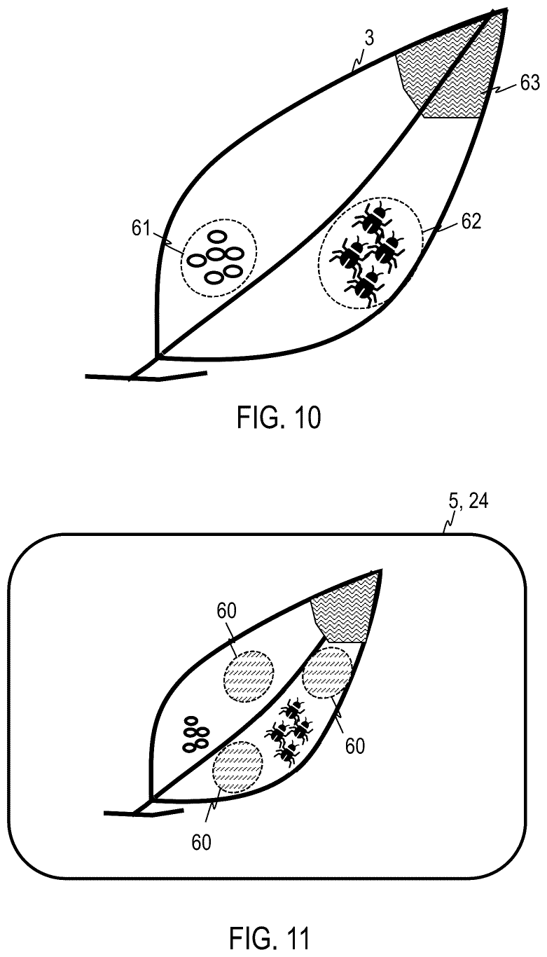

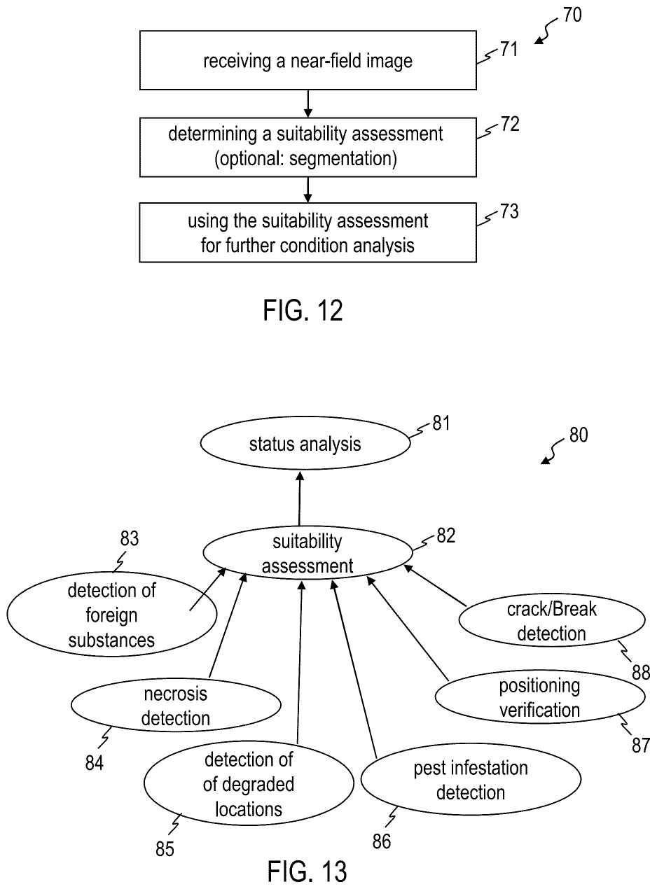

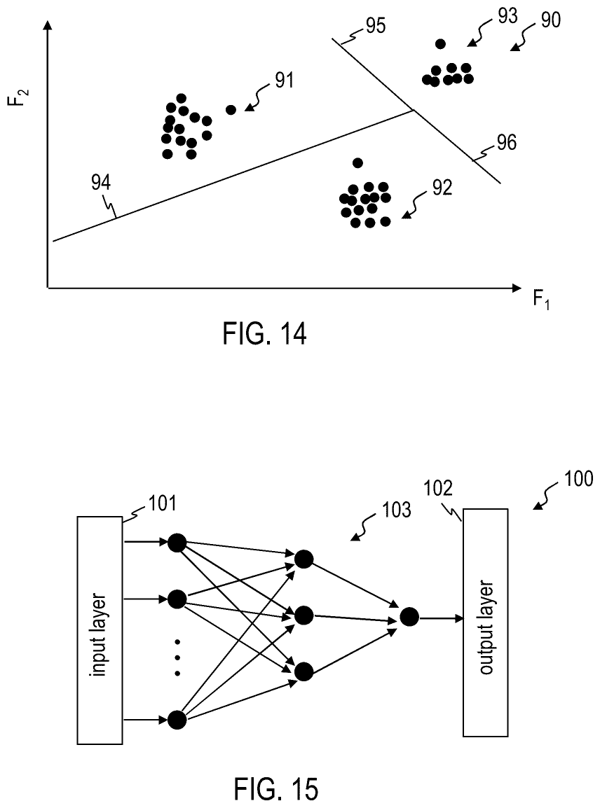

The operation of the apparatus 10 and/or the evaluation device or the evaluation system 4 during evaluation of the near-field image(s) is described in more detail with reference to FIG. 10, FIG. 11, FIG. 12, FIG. 13, FIG. 14 and FIG. 15.

FIG. 10 shows an example of a leaf 3. A leaf section of the leaf 3 is in contact with the contact surface 34 when the apparatus 10 is in use. The leaf 3 may comprise areas 61, 62 in which the acquisition of spectral analytical information for the condition analysis entails an increased risk of distortion of the analysis results due to foreign substances such as dirt, pollen or liquids or living organisms such as insects. The leaf 3 may comprise areas 63 in which, due to mechanical damage, necrosis, chlorosis, anthocyanosis or degraded leaf areas, acquisition of the spectral analytical information for the condition analysis entails an increased risk of distortion of the analysis results. The near-field image evaluation detects whether such areas and/or locations are present at the measurement spot of the spectral analytical acquisition device.

FIG. 11 shows an example of outputting the evaluation result via a display device, such as the human-machine interface 24 of the apparatus 10 and/or the display device 5. Overlaid on the near-field image, areas 60 that are suitable for the acquisition of the spectral analytical information can be marked. Alternatively or additionally, the unsuitable areas may also be marked. In this way, the acquisition of the spectral analytical information is guided in such a way that the acquisition at an unsuitable location is avoided based on objective criteria.

By using the near-field image, the more extensive condition analysis (which can be carried out in the conventional way using spectral analytical information) can thus be supported automatically and in a data-based manner.

FIG. 12 is a flowchart of a method 70. The method 70 may be performed automatically by the apparatus 10 and/or the evaluation device or the evaluation system 4.

At 71, at least one near-field image acquired by the near-field camera 21 of the apparatus 10 is received.

At 72, a suitability assessment is determined by processing the at least one near-field image. The suitability assessment may be represented by a binary value (suitable/unsuitable) for the entire plant section represented in the at least one near-field image. The suitability assessment may be a value (for example, an integer value) selected from three or several discrete values; for example, this value may indicate gradations of suitability, for example, using a value selected from an ordinal scale of values. The suitability assessment may be a value that is variable within a continuous numerical range, for example between a lower limit and an upper limit (such as 0% to 100%). The suitability assessment may also indicate which areas and/or positions on the plant portion are sufficiently intact and free of foreign substances to perform a condition analysis based on spectral analytical information collected on that plant section.

At 73, the suitability assessment is used for the more extensive condition analysis. This can be done in various ways, as explained above.

FIG. 13 is a schematic representation 80 for further illustrating the processing of the at least one near-field image. The at least one near-field image may be processed to assess the suitability, for the acquisition and/or analysis of the spectral analytical information, of a plant section positioned against the apparatus 10.

The suitability assessment 82 may be determined based on the absence of various possible sources of error. The plant section may be assessed as suitable if all verified sources of error are absent. More complex techniques may be used. For example, the plant portion may be assessed as suitable if an area percentage of the plant portion surrounded by the abutment surface 34 is greater than threshold, with the area percentage being the proportion of the plant portion that is free of all checked sources of defects.

The suitability assessment 82 has an influence on the condition analysis 81. This can be done in various ways, as has already been detailed. For example, suitability assessment 82 may be used to ensure that acquisition and/or evaluation of the spectral analytical information is performed only on plant sections assessed as suitable. Alternatively or additionally, the suitability assessment 82 may be used to mark the result of the condition analysis (for example, as reliable/limited reliability) or to assess (e.g., to weight) it.

To determine the suitability assessment, e.g., one, several, or all of the following checks may be performed using the at least one near-field image:
foreign substance detection 83: detection of liquid and/or solid foreign substances (such as dust or pollen) in the plant section positioned in the first field of view or in the second field of view;
necrosis detection 84: detection of necrotic locations in the plant section positioned in the first field of view or in the second field of view;
chlorosis detection and/or anthocyanosis detection: detection of locations with chlorosis or abnormalities with respect to anthocyanins in the plant section positioned in the first field of view or in the second field of view;
detection of degraded locations 85 in the plant section positioned in the first field of view or in the second field of view;
pest detection 86: detection of pests or insects in the plant section positioned in the first field of view or in the second field of view;
position verification 87: detection of misalignment of the apparatus on the plant. For example, automatic detection of the plant section in the first field of view or in the second field of view can be used to determine whether the apparatus is positioned on a plant section of the correct type (e.g., a leaf having a desired leaf stage);
crack/break detection 88: detecting mechanical damage in the plant section positioned in the first field of view or in the second field of view.

The inspection can be restricted to the second field of view, using position information determined in a calibration. The position information may provide the position of the second field of view relative to the first field of view. The position information may quantify an influence of a contribution of several pixels of the near field image respectively during the acquisition of the spectral analytical information. The determination of the position information in a calibration has already been described.

By limiting the check to the second field of view, the suitability assessment can be provided more quickly and can be used more quickly.

A check via the first field of view can be particularly advantageous if a position of the second field of view within the first field of view can be changed in a controllable manner.

Optionally, additional or alternative potential sources of error can be checked automatically using the at least one near-field image. For example, the at least one near-field image can be checked with regard to its consistency with user input. If the plant species or plant variety for which the condition analysis is to be performed is determined by an input that can be provided via the human-machine interface 24 or by the evaluation device or the evaluation system 4, the near-field image can be used to ensure that the apparatus 10 is positioned on a plant of the desired plant species or plant variety. If this is not the case, it is judged to be unsuitable. Alternatively or additionally, if the type of condition analysis to be performed (for example, by selecting nutrients whose concentration is to be determined based on the spectral analytical information or determining the water supply) is determined by an input that can be provided by the human-machine interface 24 or by the evaluation device or evaluation system 4, the near-field image can be used to ensure that the apparatus 10 is positioned against a plant section suitable for this type of condition analysis.

Any of the aforementioned detection tasks 83-88 may be performed using known techniques of computerized image analysis. For example, segmentation techniques may be used to detect necrotic, degraded, or mechanically damaged locations or foreign substances and pests. The segmentation techniques can use edge filters and/or color-dependent techniques. A check as to whether the apparatus 10 is positioned against a plant having the desired variety or species and/or on a plant section appropriate for the particular condition analysis (for example, a leaf having a particular leaf stage) can be performed by determining feature vectors and matching the feature vectors against a database or by other techniques that provide a comparison metric between images. Such techniques are known to those skilled in the art.

Techniques such as support vector methods (FIG. 14), which preferably convert derived features of the at least one image into a classification decision regarding the at least one source of error, can also be used to recognize the identified sources of error. Other trained machine learning models, such as neural networks (FIG. 15) or a vision transformers (ViT), can also be used to recognize the identified sources of error. The parameterization of the corresponding detection logic for detecting the various sources of error can be data-driven using training images. The training images can respectively be annotated with annotations that indicate which of the fault source(s) to be detected is/are present in the corresponding training image. The training can be carried out as supervised learning, semi-supervised learning or unsupervised learning. The parameterization of the recognition logic(s) that achieves good or even optimal performance on a test data set can be determined using known optimization methods such as gradient methods.

FIG. 14 shows an example of a recognition logic based on a support vector model. Here, images are converted into feature vectors of features F₁, F₂, wherein the application-relevant information is preferably retained and application-irrelevant information is discarded. The skilled person is familiar with suitable techniques for this, for example histograms of oriented gradients (HOG) or descriptors of the scale-invariant feature transform (SIFT). Activation features of layers of existing image classification models such as AlexNet or VGG are also possible. In this space of features, decision boundaries 94, 95, 96 can be defined with the support vector model. The feature vectors are located in different areas 91, 92, 93 of the feature space based on the respective error sources. During training, the decision boundaries and optionally also the suitable features are determined automatically before the support vector model is used. The corresponding parameters can be stored in the apparatus 10 and/or the evaluation device or the evaluation system 4 or can be stored so that they can be accessed by the latter. In use, the apparatus 10 or the evaluation device or the evaluation system 4 can evaluate the at least one near-field image. For this purpose, the feature vector for the at least one near-field image can be determined and, in relation to the decision boundaries, it can be determined in which area of the feature space the feature vector for the at least one near-field image lies and what type of error source (if any) is present. In a further embodiment, the feature vector can be used only to check whether the feature vector for the at least one near-field image lies in the area of the feature space that is assigned to the “suitable” assessment.

FIG. 15 shows a schematic representation of a machine learning model 100 that can be used to evaluate the near-field image. The machine learning model 100 comprises an input (for example an input layer) 101, one or several hidden layers 103, and an output (for example an output layer) 102. Instead of or in addition to the hidden layers 103, stacks of other processing units may also be present. The machine learning model 100 may be configured to receive, as input, pixel values of the at least one near-field image and/or data or features (for example, detected edges) derived therefrom. The trained machine learning model 100 may be trained to provide as output a value that indicates the presence or absence of a particular error source (for example, one of the error sources explained with reference to FIG. 13). The trained machine learning model 100 may be trained to provide as an output a value that represents the suitability assessment (for example, as a binary value of suitable/unsuitable).

Optionally, several trained machine learning models can be trained to check various sources of error. In this way, trained machine learning models can be trained, each of which is trained to recognize one or several possible error sources. Reliability can be further increased thereby.

The machine learning models can be trained separately for different plant species or plant varieties. This can be particularly useful for sources of defects that may be visually dependent on the plant variety or plant species. Examples of this are necrosis, chlorosis, anthocyanosis or degraded leaf spots. For sources of defects that comprise no or only a slight dependence of the visual appearance on the plant variety or plant species, a common machine learning model can be trained that is applicable to several plant varieties or plant species. An example of this is the recognition of foreign substances.

Machine learning models that are suitable for automatic image processing are known to those skilled in the art. Suitable machine learning models include, for example, so-called “convolutional neural networks” (CNN) or other so-called “deep learning” (DL) techniques. The at least one near-field image can be processed using at least one DL technique, for example CNNs or “VisionTransformer” (ViT), both of which are known to the person skilled in the art.

In a specific example, a ResNeXt50 machine learning model can be used, preferably with its parameters already pre-trained by training on another, preferably larger, data set and adopted as initialization. Depending on the recognition task with binary annotated images described below, an adaptation can be carried out separately for each recognition task:
Plant variety/plant species recognition
   Goal: Avoiding accidentally measuring plants that do not match the stored nutrient analysis algorithm
   Input data: Near-field image(s)
   Output data: Identifier of the plant variety or plant species
Leaf age detection
   Goal: Avoiding accidental measurement of leaves that do not match the required leaf age for the respective plant species
   Input data: Near field image(s)
   Output data: Leaf age as one of several possible options (e.g. classification into classes “young”, “young mature” and “mature”)
Water drop/water film detection
   Objective: Avoiding accidental measurement of leaves that would produce a distorted measurement result due to water residue
   Input data: Near-field image(s)
   Output data: Water droplets or water film present yes/no
Detecting necrotic leaf spots
   Goal: Avoiding accidental measurement of leaves that have necrotic parts
   Input data: Near field image(s)
   Output data: Leaf contains necrotic locations yes/no
Detecting degraded leaf spots
   Goal: Avoiding accidental measurement of leaves that have otherwise degraded parts
   Input data: Near field image(s)
   Output data: Leaf contains degraded locations yes/no
Aphid detection
   Goal: Avoiding accidental measurement of leaves with aphids on them, which in turn would influence the nutrient concentration of the measurement
   Input data: Near-field image(s)
   Output data: Aphid present yes/no
Detecting pollen/dust/foreign objects
   Goal: Avoiding accidental measurement of leaves that have additional (non-living) foreign objects that would falsify the measurement
   Input data: Near field image(s)
   Output data: Leaf contains pollen/dust/foreign bodies yes/no
Detecting twisted/kinked leaf
   Goal: Avoiding acquisition of spectral analytical information partially or completely on an abaxial side (if acquisition on the adaxial side is desired) or on the adaxial side (if acquisition on the abaxial side is desired), which could lead to erroneous results (e.g. different texture of the reverse side)
   Input data: Near field image(s)
   Output data: Leaf twisted/bent yes/no
Detecting wrong side of the leaf
   Objective: Avoiding the acquisition of spectral analytical information on the reverse side of the leaf, which could lead to incorrect results (e.g. different texture of the reverse side)
   Input data: Near-field image(s)
   Output data: Wrong leaf side yes/no
Detecting broken/torn leaf
   Goal: Avoiding scanning the background in locations where the leaf is torn
   Input data: Near field image(s)
   Output data: Broken/torn leaf yes/no
Incorrect positioning
   Aim: Checking correct positioning of the sample under a measurement window
      Example: Too close to the edge of the leaf, not filling the entire measurement window
   Input data: Near-field image(s)
   Output data: Incorrectly positioned yes/no
Correct contact pressure of the leaf on a sample holder of apparatus 10
   Aim: Checking correct leaf contact pressure of the sample on the measuring window
      Example: For example, protruding curved leaves on the glass
   Input data: Near-field image(s)
   Output data: not pressed on correctly, or too much object-air distance, or too strongly curved leaf

For each of these described recognition logics, a DL (“deep learning”) model can be provided, for example, that predicts an output value for each possible detection state in relation to the corresponding error source. For example, the prediction of two values for binary yes/no cases, or several for a classification task in more than two possible classes. The parameters of the DL model can be determined iteratively using known optimization techniques, for example, with respect to known target metrics such as cross entropy with annotated training data.

The recognition logic may also be configured as a detection model (preferably as a DL detection model) so that, for example, the positions for the at least one near-field image are determined using parametric descriptions such as bounding boxes, which indicate the presence of sources of defects. An example is the detection of all aphids present on the leaf in the current (first or second) field of view to detect an aphid infestation of the sample. In a specific example, a RetinaNet machine learning model can be used.

The recognition logic can also be configured as a segmentation model (preferably a DL segmentation model), so that, for example, the positions that indicate the presence of error sources are determined with pixel accuracy for the at least one near-field image. An example is a semantic segmentation of all pixels in the current (first or second) field of view where pollen is present. In a specific example, a Mask2Former machine learning model can be used.

When training the machine learning model or machine learning models, augmentation techniques may be applied to artificially generate more images available for training. Augmentation may comprise one, several or all of the following operations that can be applied to annotated images: random cropping, random rotations, random flipping (left-right and/or top-bottom), random changes in brightness, random changes in contrast.

To perform training and testing, the annotated images (advantageously before augmentation) can be divided into a training data set (e.g., 70% of the images), a validation set (e.g., 15% of the images) and a test set (e.g., 15% of the images). Especially when training classifiers, it is advantageous to split the training, validation and test set separately for each annotated class. This is especially advantageous for classes with very unequal amounts of images.

Augmentation can be used to reduce or eliminate imbalances in the number of annotated images belonging to different classes. Alternatively, a selection from a numerically underrepresented class can be made more frequently during training to use images from all classes at least approximately equally often during training.

For a machine learning model that is to perform detection (e.g., an aphid detector), it is advantageous for the annotated images to comprise at least one hundred images of a plant species with the object to be detected. The images can show different leaf stages. For an aphid detector that can be applied to several plant species, at least ten further images with the object to be detected can be used in the annotated training data for each additional plant species. In this way, a general detector (applicable to several plant species) can be trained (for example, a general aphid-on-leaf detector).

For a machine learning model that is to perform segmentation (for example, a pollen segmenter), it is advantageous for the annotated images to comprise at least twenty-five images with pixel annotations. It is advantageous if some images do not contain pollen, but contain visually similar symptoms (such as burnt leaf spots). Optionally, some further images (for example at least five) may contain only leaves, but no pollen and no visually pollen-like symptoms. Optionally, further images may comprise other known disturbances (such as a kink, water on the leaf, . . . ). This training data can be used for the segmenter for the first plant species.

If an application to further plant species is desired, advantageously at least five pixel annotated images with pollen from each further plant species can be used in the training data set. This allows a general pollen-on-leaf segmentation model to be trained.

For a machine learning model that is to perform classification (for example, water film classification), the training data can advantageously comprise at least 1000 images, of which at least 250 show a leaf with a water film. The images can be distributed over several growth stages (plant and/or leaf stages).

Further improvements can be achieved with correspondingly larger amounts of training data. For example, it may be advantageous to use at least five times, at least ten times or at least approximately twenty times the number of images mentioned in the previous paragraphs during training. As explained, a division into training, validation and test sets is performed.

The results of the different recognition techniques can be logically combined with each other. One or several filters can be used to implement the recognition logic. For example, an assessment as unsuitable can be made if at least one aphid was detected during aphid detection or if more than a certain area (e.g. 2 mm²) is covered by pollen for a segmentation that determines pollen-covered areas.

An assessment as suitable may be made if a certain possible disturbing factor (for example aphid infestation) is present within the first field of view 41, but not in the possibly smaller second field of view 42.

Through the acquisition and evaluation of the at least one near-field image, the condition analysis can be avoided in the presence of unsupported fault cases. This avoids a risk of incorrect operation of the apparatus 10, based on objective criteria. Expert knowledge in the field operation of the apparatus 10 is no longer necessarily required. Fault detection is partially or fully automated.

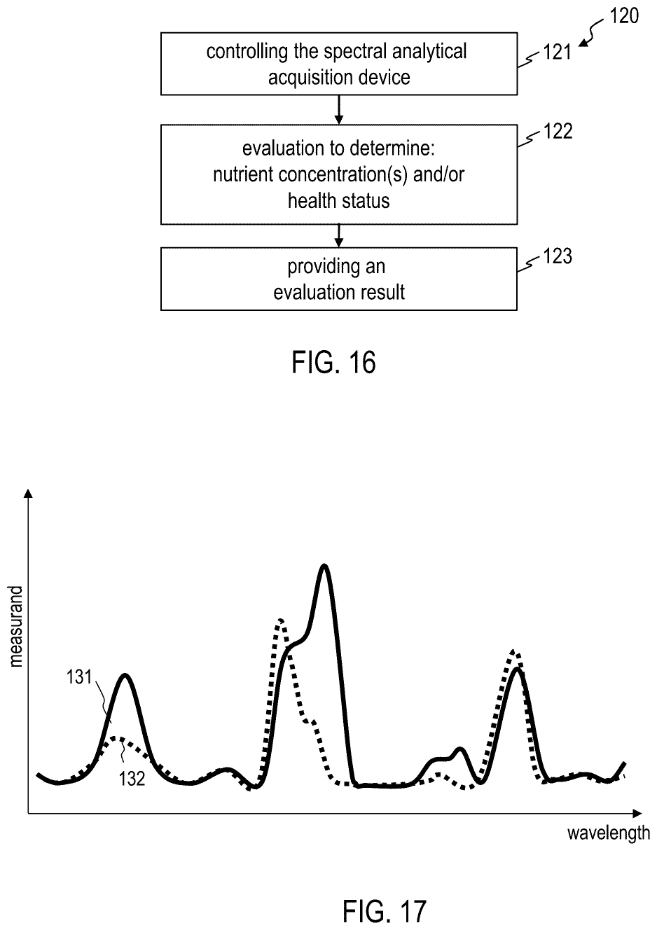

FIG. 16 is a flowchart of a procedure 120 that may be used to utilize the evaluation result of the at least one near-field image. The procedure 120 may be used to implement step 73 of the method 70 of FIG. 12. The procedure 120 may be automatically performed by the apparatus 10 and/or the evaluation device or the evaluation system 4.

At 121, the spectral analytical acquisition device is controlled. The control can be based on the evaluation result of the at least one near-field image, as has already been explained.

At 122, acquired spectral analytical information is evaluated. Thereby, a nutrient concentration of at least one and advantageously several nutrients can be determined and/or the health status of the plant can be assessed.

At 123, the evaluation result is provided. This can be done locally at the apparatus 10 or via the evaluation device or the evaluation system 4. The evaluation result can be used to adjust the cultivation condition of plants so that a good, preferably optimal, supply is achieved.

FIG. 17 shows exemplary spectral analytical information, respectively configured as a spectrum 131, 132 over a wavelength range. The wavelength range can extend over at least part of the visible and/or near-infrared spectrum. For example, the wavelength range may be within a range from 300 nm to 2000 nm, or from 500 nm to 1650 nm, optionally also covering these ranges.

The spectral analytical information may comprise an optical measurand (for example, an intensity of reflected, scattered or Raman-scattered electromagnetic radiation or fluorescence radiation) as a function of wavelength for several wavelengths.

The spectral analytical information varies as a function of the concentration of various nutrients. For example, an undersupply of some nutrients leads to a reduction in local maxima (“peaks”) characteristic of these nutrients. This can be used to quantitatively determine the concentration of various nutrients, an under-or oversupply of water or other health status characteristics of the plant based on the spectral analytical information.

The condition analysis based on the spectral analytical information is affected by the at least one near-field image. In particular, the at least one near-field image can be used to avoid errors due to acquisition of the spectral analytical information at an unsuitable location of the plant.

The processing of the spectral analytical information to determine the health status can be performed in various ways. For example, the techniques described in WO 2019/169434 A1, US 7 804 588 B2 and US 11 320 307 B2 can be used. In general, in a phase in which the processing logic for processing the spectral analytical information is parameterized, laboratory test results determined on the respective leaves can be used in addition to the spectral analytical information in order to correlate the spectral analytical information with nutrient concentrations or other condition data.

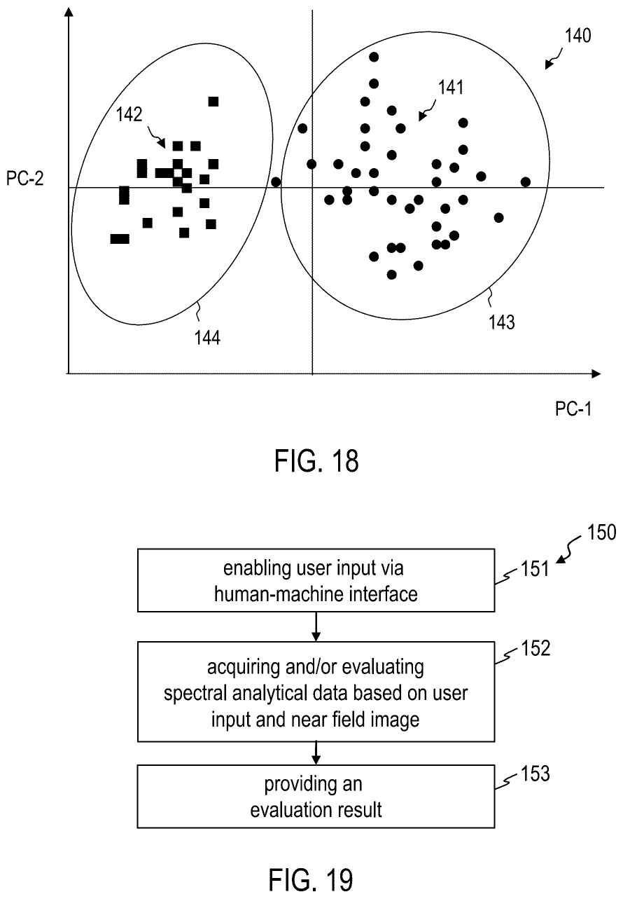

Alternatively or additionally, techniques such as cluster or principal component analysis can be used. FIG. 18 illustrates this as an example for a principal component analysis 140, where principal components can be determined from the respective acquired spectrum and their association with different areas 143, 144 in a space spanned by the principal components can be determined. Depending on the nutrient concentration, the principal components lie in an area in which spectra for plants with similar nutrient concentrations come together in clusters 141, 142. The nutrient concentrations for each cluster 141, 142 can be quantitatively determined in a training phase prior to field use of the apparatus 10. In addition to the spectral analytical information, laboratory test results determined on the respective leaves can be used to correlate the spectral analytical information with nutrient concentrations or other condition data.

Alternatively or additionally, machine learning techniques can be used to infer quantitative results on nutrient concentration(s) and/or other factors influencing the plant condition (such as water supply) from the spectral analytical information. For example, a least squares regression method, a PLS regression method, a support vector regression, a Gaussian process regression, a k-nearest neighbor method, a random forest regression, multilayer perceptrons and/or CNNs (“convolutional neural networks”) can be used to determine the nutrient concentrations. At least one machine learning model can be trained to receive the spectral analytical information as input and to output one or several nutrient concentration(s) as output. When training the at least one machine learning model, spectral analytical information is used that is annotated with the laboratory-determined nutrient concentration for one or several nutrients. ML classification techniques may also be employed, the output of which predicts an association of the nutrient concentration to two or several different value ranges. For example, these can represent normal supply, undersupply and oversupply of the respective nutrient. The machine learning models can then be trained accordingly so that they do not regress on a quantitative nutrient concentration, but instead provide, as a classifier, the assignment to possible conditions such as normal supply, undersupply and oversupply. For example, support vector machines, a k-nearest neighbor model, a “Random Decision Forest” model, MLPs, CNNs or other models can be trained as classifiers.

For a time-efficient execution of the condition analysis, it is possible for various parameters to be predefined by the user. For example, the apparatus 4 or the evaluation device or the evaluation system 4 may be configured to allow the user to specify the plant species or plant variety and nutrients of interest via a human-machine interface. The evaluation of the near-field image and/or the condition analysis based on the spectral analytical information can then be carried out based on the user-defined plant species or plant variety and the nutrients to be determined.

FIG. 19 is a flowchart of a method 150. The method 150 may be performed automatically by the apparatus 10 and/or the evaluation device or the evaluation system 4.

At 151, a human-machine interface is controlled to enable a user input. The user input may, for example, specify the plant species or plant variety and nutrients of interest. Alternatively or additionally, the user input may determine which checks are to be performed using the at least one near-field image and prior to acquisition of the spectral analytical information can occur.

At 152, spectral analytical data is acquired based on the user input and based on the evaluation of the at least one near-field image. For illustration, the spectral analytical acquisition device may be controlled such that the spectral analytical information is selectively acquired only if the evaluation of the near-field image shows that the plant section positioned against the apparatus is suitable for determining the concentration of the specified nutrients from the spectral analytical information.

At 153, the evaluation result is provided. This can be done via the human-machine interface 24 of the apparatus 10 or a human-machine interface of the evaluation device or the evaluation system 4. The provision of the evaluation result can cause an automatic or semi-automatic adjustment of cultivation conditions.

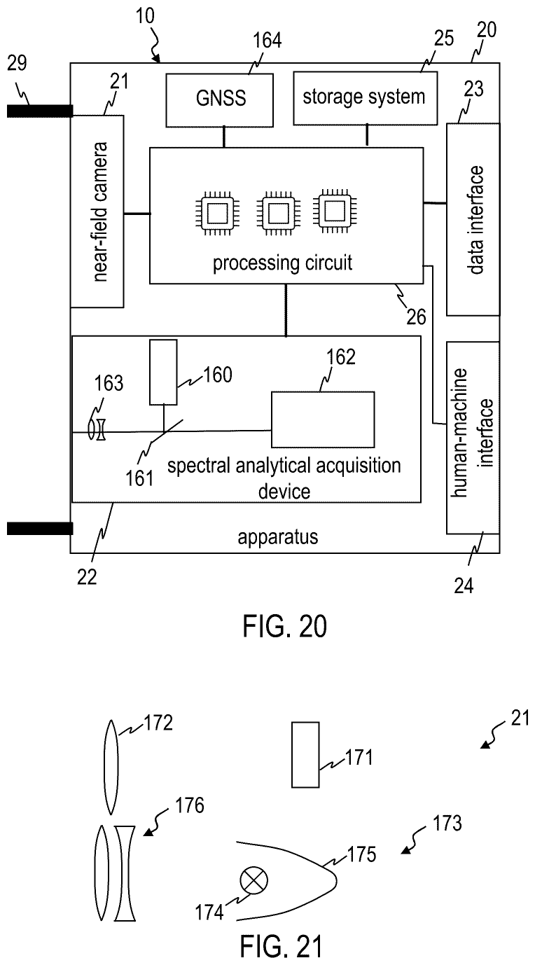

FIG. 20 shows additional features of the apparatus 10 that can be used with the apparatus, methods and/or systems disclosed herein.

The spectral analytical acquisition device 22 of the apparatus 10 may comprise a sample illumination source 160, a spectrometer 162, and optical components 161, 163. The optical components 162 may comprise at least one controllable component 161. The spectral analytical acquisition device 22 can be configured such that the second field of view 42 of the spectral analytical acquisition device 22 is adjustable by control of the controllable component 161. The adjustment can be performed based on the at least one near-field image.

The apparatus 10 may comprise a positioning system for determining the position of the apparatus 10 in a reference system. The positioning system may comprise a global navigation satellite system (GNSS) 164. The apparatus 10 may be configured to determine the position of the apparatus 10 using the positioning system during an acquisition of the at least one near-field image and/or the spectral analytical information. The position that is respectively determined can be used to determine a correlation between plant position and the respective plant condition when analyzing the condition of several plants in a region.

FIG. 21 is a schematic representation of a configuration of the near-field camera 21. The near-field camera 21 comprises a camera chip 171 (for example a CMOS or CCD chip) and a near-field optical system 172, which is only shown schematically. The apparatus 10 may be configured to provide active illumination during acquisition of the at least one near-field image. An illumination device 173 of the apparatus 10 may comprise a light source 174, which can be selectively activated during acquisition of the at least one near-field image. Illumination optics, which may comprise a paraboloid or facetted reflector 175 and other optical components 176, may provide illumination, for example with parallel light.

The near-field optics 172 can be adaptive. Alternatively or additionally, the apparatus 10 may comprise several near-field optics, from each of which at least one may be selected for the acquisition of the at least one near-field image.

With reference to the drawings, apparatus, evaluation devices and evaluation systems, systems and methods have already been described in detail, which determine information about a sample status and use it in the condition analysis of plants. A near-field camera can be used that has a short focal length objective for near-field imaging and that includes an illumination optics unit for sample illumination, image processing techniques for automatically analyzing the captured near-field images, a computing unit for executing the image processing techniques and a storage system for holding the learned image processing techniques.

In the following, various other modifications and features are below, which can optionally be employed to support the technical effects previously described.

Field of view: The first field of view 41 can essentially correspond to the second field of view 42. A larger field of view 41 of the near-field camera compared to the measuring range of the spectral analytical acquisition device has the advantage that the illuminated area of the spectrometer can be moved unintentionally during the measurement and the (more global) camera analysis remains valid. Furthermore, leaf changes at the edge of the measuring range of the spectral analytical acquisition device are also recognizable.

Color channels: A color camera image (e.g. RGB-color channels) of the near-field camera can be advantageous, for example to assess the plant leaf stage or condition or to distinguish between color inhomogeneities and glare over the leaf surface. The camera image may comprise several color channels in the visible spectral range and one or several color channels in the near-infrared spectral range.

Configuration of the illumination optics: The illumination optics 175, 176 for the sample illumination should preferably be configured such that the light beams of the light source/lamp burner impinge on the sample as parallel beams and are picked up by the camera optics due to the sample reflection. This can be realized by paraboloid and/or facetted reflectors or the light source acts as a point light source in the sample measurement plane.

Camera calibration: Camera calibration or camera referencing can be performed using a diffuse white/grey standard during device initialization (one-time or recurring). With recurring white/grey standard measurements, intensity and/or spectral changes in the light source can be detected and compensated for. The apparatus according to any of the embodiments of the invention disclosed herein may comprise an integrated white standard (for example from Spectralon) for adjusting the channel amplification factors (white balance) and adjusting the exposure time.

Enabling user-defined input of analyses to be performed: Every analysis takes time. Some users do not want to perform all possible sources of error and take the risk of accidentally measuring a non-supporting sample in order to achieve faster measurement times. Therefore, it may be advantageous for users to specify which tests are to be performed based on the at least one near-field image and how precisely they are to be performed. For example, a human-machine interface can be controlled to enable user input that indicates the recognition tasks to be performed (e.g., only a check for correctness of the plant variety before spectral analysis data acquisition).

Visualization of affected areas in the near-field image: In the case of a necessary repeat measurement, it can be helpful to visually display to the user which areas of the image have been classified as degradation-affected. This can be derived from the analysis of the machine learning models, for example. User support can be provided by visualizing the areas that were relevant for the analysis result—for example with techniques familiar to the expert such as GradCam/XRAI, etc.

Determination of defects by pixel or area in the near-field image: It is also possible to perform a “spatial” analysis of which areas in the image can be attributed to the respective problem classes, which is possible, for example, as a pixel-accurate segmentation. This has several advantages. A better visualization for the user can be attained. More precise control is possible, for example by defining maximum areas that may have certain undesired characteristics. These advantages can outweigh the additional effort in terms of annotation in the training phase and longer computing time. Thus, the classification need not relate to the plant part in the first or second field of view in its entirety, but may also relate to pixels or areas contained therein.

Aggregation over several positions of the apparatus 10: The disclosed techniques have so far been described primarily with respect to determining a suitability assessment for a condition analysis of a single plant. Evidently, the techniques can be used repeatedly or, when using several apparatus 10, in parallel on different plants, for example in a cultivation area. In this case, the results from several recording positions can be aggregated and further processed in relation to a reference system (for example a field, a forest area, or another cultivation area). For example, a frequency of positive aphid analyses over an entire field and/or a frequency of leaf necrosis and/or chlorosis and/or anthocyanosis can be considered, which may indicate a locally poor growing medium. Positioning system 164, for example a GPS system or other GNSS, may be used for spatial mapping.

Output (e.g. visualization) of uncertainties: In addition to predicting the suitability for the acquisition of the spectral analytical information, an (un)certainty of the analysis of the at least one near-field image may also be determined and used. The uncertainties can be determined in various ways and output as qualitative or quantitative uncertainty measures. For example, the uncertainties can be determined based on initial values of classifiers, based on uncertainties determined during training, based on a spread of results of image analysis with several trained machine learning models, based on a spread of nutrient concentrations determined on different leaves or different plants based on images, or in other ways. The uncertainties can be output via a human-machine interface and/or used to automatically control the spectral analytical acquisition device.

Some or all of the image evaluation steps may comprise a determination of a confidence or uncertainty for the evaluation result, i.e. the uncertainty of or confidence in the suitability, for the acquisition of the spectral analytical information, determined based on at least one near-field image. The confidence or uncertainty can be determined, for example, using one, several or all of the following techniques:
An uncertainty can be derived directly from the output values of a machine learning model. For example, in a classification procedure, entropy can be used as a measure of uncertainty via single class probabilities. Alternatively, the difference between the safest and second safest class can be used as a measure of uncertainty.
An uncertainty measure can be determined by estimating the similarity of the at least one image to the training data. For example, so-called “activation shaping” for CNNs (“convolutional neural networks”) can be used for this purpose. Separate models can also be used to determine anomalies or deviations.
Uncertainties can be determined by mapping them to the uncertainty of a test data set (during training). For example, the mean error on a test data set can be determined and used as the expected uncertainty for each output value determined using the machine learning model.

The above techniques can be used in particular for evaluations that solve classification tasks.

For detection tasks, for example, the confidence or uncertainty can be determined by the machine learning model having an output that also outputs a confidence value. The machine learning model can be trained and used in such a way that, in addition to a detection result, it also outputs the associated confidence or uncertainty.

For segmentation tasks, the confidence or uncertainty can be determined using one or several of the following techniques, for example:
An uncertainty can be derived directly from the output values of a machine learning model. For example, entropy can be averaged over single class probabilities for all pixels in a region of equal class membership.
An uncertainty can be derived from a variation of the output values with repeated, random variation of the input image (augmentation during the application or so-called “test time augmentation” techniques) or from intermediate calculations (for example by so-called “test time drop-out” techniques).

Alternatively or additionally, for detection tasks and/or segmentation tasks, a measure of uncertainty can be determined by estimating the similarity of at least one image area (e.g. a region or box) to image areas in the training data. It is also possible to use separate models to determine anomalies or deviations.

The results of the image evaluation steps can be filtered or weighted based on the associated confidence or uncertainty. For example, only those results can be used further (e.g. for visualization) that have a confidence exceeding a threshold. It is also possible that only the k most reliable results are output. The threshold or the number k can be user-defined. For example, in the visualizations shown in FIG. 11, only those areas whose confidence is above a threshold or which belong to the k most reliable results can be visualized.

Configuration and mounting of the apparatus 10: The apparatus 10 can be configured as a portable, in particular handheld, apparatus 10.

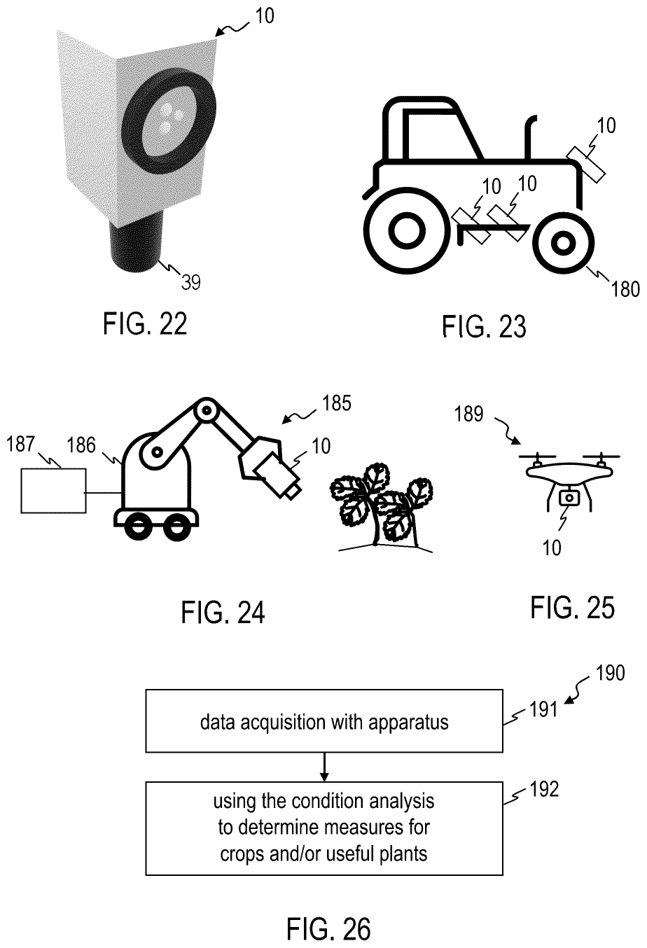

FIG. 22 schematically shows an embodiment of the apparatus 10 as a handheld device comprising a structure 39 for holding the apparatus 10.

FIG. 23 shows a further embodiment in which a vehicle 180 according to an embodiment comprises an apparatus 10 mounted thereon, or several apparatuses 10 mounted thereon. Accordingly, the apparatus 10 or the apparatuses 10 may be configured to have a retaining structure for attachment to an agricultural engineering vehicle.

FIG. 24 shows a further embodiment in which a robot 185 comprises the apparatus 10. A base 186 of the robot 185 may be stationary or mobile, for example along a rail system. A controller 187 may control one or several actuators for positioning the apparatus 10.

FIG. 25 shows a further embodiment in which an aerial vehicle 189 comprises the apparatus 10. The aerial vehicle 189 may be remotely controllable and/or configured for autonomous flight operation.

In still another embodiment, the apparatus 10 may be configured as a stationary system. The apparatus 10 may be mounted by means of a stationary bracket in a vicinity of a conveyor on which plants are conveyed past the apparatus 10.

Evaluation of the at least one near-field image: The evaluation of the at least one near-field image can be performed in different ways, as already explained. In particular, classifiers can be used to distinguish defective from defect-free plant sections. A large number of recognition techniques (e.g. CNN, ViTs, support vector machines with, for example, features defined on the basis of expert knowledge, without being limited thereto) can be used. Various techniques can also be used for training, for example gradient-based optimization techniques such as stochastic gradient descent (SGD) or an optimization technique with adaptive learning rate (such as ADAM), fully supervised, semi-supervised and other training techniques.

Spectral analytical acquisition device: The spectral analytical acquisition device 22 may comprise a spectrometer. However, other embodiments are possible that allow the acquisition of an optical measurand at two or several (for example, at least ten or more) wavelengths. For this purpose, filter-based systems (which are configured to block light components), single wavelength diodes or other implementations can be used as an alternative or in addition to the use of a spectrometer.

Ways of using the evaluation of the at least one near-field image for the acquisition and/or evaluation of the spectral analytical information: The at least one near-field image and its evaluation (in particular the suitability assessment) can be used in various ways for the acquisition, evaluation or assessment of a condition analysis based on spectral analytical information. This includes, for example, the following techniques:
Using the at least one near-field image to decide whether or when to acquire the spectral analytical information: Here, only near-field images may be acquired initially and evaluated continually or in response to a trigger signal to assess the presence of faults. The spectral measurement is carried out only if the evaluation shows that the sample condition is as intended. The result can be output, for example via an optical, acoustic and/or tactile signal. Capturing a near-field image before the spectroscopic measurement can save time if an anomaly is detected, as the unnecessary calibration step and spectroscopic measurement step can be omitted.
Use of the at least one near-field image to trigger a user confirmation: If the evaluation of the near-field image indicates that there is an issue, a human-machine interface can be controlled to enable a user confirmation. The spectral analytical information can be acquired selectively only if the user confirmation via the human-machine interface confirms that the spectral analytical information should be acquired despite the issue.
Evaluation of the at least one near-field image after acquisition of the spectral analytical information: In this embodiment, the apparatus 10 is configured such that the spectral analytical information is acquired independently of the evaluation of the at least one near-field image. The spectral analytical information can optionally also be analyzed independently of the evaluation of the at least one near-field image. The spectral analytical information and the near-field image can be evaluated independently of each other. Both results can be provided to the user, for example as a result of the condition analysis in combination with an indication of the expected reliability of the condition analysis determined from the at least one near-field image. For this purpose, for example, a traffic light-like system can be used to output the reliability via the human-machine interface 24 or display device 5. Alternatively or additionally, a visualization of the issue found in the sample (e.g. “aphid in the field of view”) can be provided.
Using the evaluation result of the at least one near-field image at a later time: The evaluation result of the at least one near-field image can also be used or even determined after the measurement. This allows, for example, subsequent filtering out or marking of invalid measurements from statistics for historical analyses or for analyses that aggregate results from individual measurements.

Fields of application: The disclosed apparatus, evaluation devices and evaluation system, systems and methods can be used for various applications. In particular, the apparatus, evaluation devices and evaluation system, systems and methods can be used on useful plants or cultivated plants. In particular, the plants on which the techniques can be used according to the invention may comprise plants of forestry technology (for example trees), hybrids and/or genetically modified plants.

Examples of such plants may include:
crop plants
fodder plants
fiber plants
oil plants
ornamental plants
industrial crops, such as
   oilseeds
   tobacco
   hemp
   hops
   aromatic, culinary, and medicinal plants
   seeds for herbaceous oil plants
   seeds for linseed (and consequently fiber flax)
   energy crops
   plants used for the production of feedstock for renewable energy production

The condition analysis may comprise, but is not limited to, the qualitative and/or quantitative determination of nutrient concentrations. For example, the condition analysis may comprise analyses comprising one, several, or all of the following plant conditions:
Nutrient supply problems, for example
   nutrient deficiency
   toxicity
Biotic stress, for example due to
   nematodes
   insects
   Arachnids
   Fungi
   bacteria
   viruses
Abiotic stress, for example due to
   drought
   excessive water supply and/or waterlogging
   salinity
   temperature-related stress (cold, frost, heat)
   UV light
   metal toxicity
   mechanical stress (e.g., pressure, crushing, pressing).

The result of the evaluation of the at least one near-field image and the condition analysis carried out on the basis of the spectral analytical information can be used to improve conditions for the plants. This can be done by automatic or semi-automatic adjustment of artificially supplied substances (such as approved fertilizers or approved insecticides) and human-initiated supplied water. Numerous other applications are possible, for example to monitor the health of plants with regard to environmental, including regulatory, issues.

Further areas of application: Although embodiments have been described in the context of analyzing the condition of plants, the apparatus, evaluation devices and evaluation systems and methods can also be used in other ways, for example for image-based determination of the suitability of an agricultural product or of living organisms for the acquisition of spectral analytical information for further analysis.

FIG. 26 is a flowchart of a method 190 that may be performed using the apparatus 10.

At 191, data is acquired using the apparatus according to the invention. The data can be evaluated by the apparatus 10 and/or the evaluation device or the evaluation system 4.

At 192, a result of the evaluation is used to determine measures to improve conditions for the plants. The measures may then be implemented to improve the conditions.

The disclosed apparatus, evaluation devices and evaluation system, systems and methods provide various technical effects. In particular, they provide improvements with respect to the acquisition, transmission and/or evaluation of spectral analytical information for analyzing the condition of plants. The methods, apparatus and systems can be used to support an acquisition, transmission and/or evaluation of spectral analytical information on crops or useful plants.

Embodiments of the invention can be used in particular in association with plant cultivation in agriculture, which is a central economic or industrial sector of humanity. Global trends such as the increasing population and the simultaneously decreasing availability of agricultural land are leading to a constantly growing demand for crop yields per area. At the same time, the unsustainable exploitation of soils is leading to an increased need for targeted fertilization in order to maintain or even increase previous yields. In addition, a growing awareness of sustainable farming and increasing regulatory pressure are leading to new requirements for precise fertilizer application, particularly in order to avoid over-fertilization.

A suitable supply of nitrogen (N) is particularly relevant for the growth of every crop, as this is the most important element of chlorophyll and is therefore essential for optimal metabolism. In addition to N, other macronutrients are also relevant, such as phosphorus (P) and potassium (K), which together with N can often be added to the soil as so-called NPK fertilizers, as well as calcium (Ca), magnesium (Mg) and sulphur (S). Mg is the central element in the chlorophyll ring.

In addition, possibly based on the type of plant, other nutrients may also be relevant for optimal growth and yield, but these are often required in much smaller quantities and are therefore also referred to as micronutrients. These include, for example, boron (B), molybdenum (Mo), copper (Cu), manganese (Mn), zinc (Zn), iron (Fe) and chlorine (Cl).

Embodiments of the invention can help to ensure that optimal conditions with regard to the nutrient supply are established throughout the growth phase of a plant. This is the only way to react appropriately to nutrient deficiencies or nutrient oversupply by means of adjusted application of fertilizer (by means of a specific fertigation program in the substrate or soil growth or as additional soil and/or foliar fertilization). Embodiments of the invention allow regular monitoring of plants. Compared to laboratory leaf analysis, the condition analysis can be carried out quickly. This is particularly desirable for fast-growing plants and plant species having rapid fruit ripening (for example strawberries and lettuce). Embodiments of the invention thus also address the objective that there should not be several days between measurement and availability of the evaluation result, as otherwise the measurement result is already invalid again and does not allow any meaningful adjustment of the nutrient application. Rapid evaluation is also desirable if the environmental factors change quickly (e.g. temperature, precipitation, etc.), as otherwise the condition between the measurement and the time of analysis would change considerably

Embodiments of the invention allow data collection for nutrient analysis at the intended point of use without the need to transfer physical samples. Embodiments of the invention address the fact that, in field operation, there can be several interfering factors as compared to a laboratory scenario. Examples include:
the presence of animals on the leaves (e.g., aphids, tripes, mites),
the presence of water on the leaves (rain, water film, dew, fog),
measuring leaves of the wrong plant species,
measuring leaves of the wrong leaf age,
measuring curled leaves,
measuring the wrong leaf side (usually the underside of the leaf),
measuring in a locally inhomogeneous and unrepresentative area of the leaf blade (e.g. due to fertilizer or pesticide burns or plant spotting),
measuring contaminated leaf blades (e.g. covered with pollen or soiling),
measuring over an area largely covered by midribs or lateral ribs, and/or
measuring small leaves whose coverage of the measuring window is difficult to assess with the naked eye
the measurement of degraded leaves.

Embodiments of the invention allow a nutrient analysis to be performed which is configured in such a way that the user has as little influence as possible on an invalid measurement result. Embodiments of the invention provide measures for an error-free, robust measurement with enhanced repeatability and user-friendliness. Embodiments of the invention provide in particular measures that ensure that the susceptibility to errors is reduced when testing the leaf to be measured, so that ultimately only suitable leaves are used for the nutrient analyses.

Embodiments of the invention may thus serve to provide a non-destructive, automated and simultaneously rapid (near real-time) solution for detecting deficiencies and/or excesses of several nutrients in plants, the solution being particularly user-friendly and comprising a high repeatability.

While embodiments have been described with reference to the drawings, variations can be implemented in further embodiments. For example, the modifications already described can be used cumulatively or alternatively. The evaluation device 4 does not necessarily have to be provided separately from the apparatus 10, but can also be structurally connected thereto or integrated into the apparatus 10.

While embodiments have been described that are usable with useful plants or cultivated plants, the disclosed techniques can also be used in other fields of application.

The present disclosure also includes embodiments with any combination of features mentioned or shown with respect to various embodiments. It also includes individual features in the drawings, even if they are shown in association with other features and/or are not mentioned above or below. The alternatives of embodiments described in the drawings and the description and individual alternatives of their features may also be disclaimed from the subject matter of the invention or from the disclosed subject matter.

The expressions “comprising” and “comprising” and derivatives thereof indicate a non-exhaustive relationship and do not exclude the presence of other elements or steps. The indefinite article “a” or “an” and derivatives thereof do not exclude the presence of a plurality of the corresponding elements. The functions of several features listed in the claims may be fulfilled by one unit or step.

A machine-readable instruction code, which may be executed by a programmable circuit to perform methods according to embodiments, may be stored and/or distributed on a suitable medium, such as an optical storage medium or a fixed medium provided together with or as part of other hardware. The instruction code may also be distributed in another form, such as in the form of a modulated data signal sequence.

Embodiments of the invention provide improved techniques for a condition analysis of plants.

1. An apparatus for a condition analysis of a plant, comprising:
a housing;
a near-field camera configured to acquire at least one image of at least a part of the plant;
a spectral analytical acquisition device configured to acquire spectral analytical information of the plant, and
at least one interface configured to output the at least one image and the spectral analytical information or data derived therefrom;
wherein the near-field camera has a first field of view relative to the housing;
wherein the spectral analytical acquisition device has a second field of view relative to the housing and
wherein the second field of view is completely contained within the first field of view.

2. The apparatus according to claim 1, wherein the apparatus is configured such that a suitability assessment of the area of the plant represented in the at least one image for acquisition or evaluation of the spectral analytical information can be determined based on the at least one image.

3. The apparatus according to claim 2, wherein the spectral analytical acquisition device is configured to perform the acquisition of the spectral analytical information selectively depending on the suitability assessment or the apparatus is configured to transmit or analyze the spectral analytical information or the data derived therefrom via the interface selectively depending on the suitability assessment.

4. The apparatus according to claim 2, wherein the suitability assessment comprises a suitability of different positions or areas of the plant for a determination, based on the spectral analytical information, of nutrient concentrations or a health status, wherein the apparatus is configured such that the spectral analytical acquisition device performs the acquisition of the spectral analytical information depending on the suitability of different positions or areas.

5. The apparatus according to claim 1, wherein the apparatus is configured to receive the suitability assessment via the at least one interface.

6. The apparatus according to claim 1, further comprising:
a device for reducing ambient light effects.

7. The apparatus according to claim 1, wherein the apparatus is configured as a mobile apparatus (10).

8. An evaluation device or system for a condition analysis of a plant, comprising:
at least one evaluation device interface configured to receive at least one image of at least a part of the plant; and
an evaluation circuit (45) configured to determine, based on the at least one image, a suitability assessment of an area of the plant represented in the at least one image for an acquisition or evaluation of spectral analytical information.

9. The evaluation device or system according to claim 8, wherein the evaluation circuit is configured to recognize, for determining the suitability assessment, one or more conditions selected from a group consisting of: presence of foreign substances, necrosis, chlorosis, anthocyanosis, degraded plant areas, pest infestation, misalignment of the area of the plant during image acquisition, crack or break in a part of the plant.

10. The evaluation device or system according to claim 8, further comprising a human-machine interface configured to receive a user input specifying one, more or all of the following:
plant stage,
leaf stage,
plant species,
plant variety,
at least one nutrient to be analyzed,
at least one disease to be tested,
wherein the evaluation circuit is configured to perform the suitability assessment depending on the indication or indications contained in the user input.

11. The evaluation device or system according to claim 8, wherein the at least one evaluation device interface is configured to output the suitability assessment for use in the acquisition of spectral analytical information.

12. The evaluation device or system according to claim 8, wherein the evaluation circuit is configured to perform the suitability assessment without using spectral analytical information, or wherein the evaluation circuit is configured to evaluate the at least one image to determine the suitability assessment using at least one trained machine learning model configured to receive, as input, pixel values of the at least one image or values derived therefrom.

13. The evaluation device or system according to claim 8, wherein the evaluation device interface is configured to receive spectral analytical information of the plant or data derived therefrom, and wherein the evaluation circuit is configured to perform at least one nutrient analysis based on the spectral analytical information or the data derived therefrom.

14. A system, comprising:
the apparatus according to claim 1, and
the evaluation device or evaluation system according to claim 8.

15. A method for a condition analysis of a plant comprising:
receiving at least one image of the plant;
determining a suitability assessment of the plant for an acquisition or evaluation of spectral analytical information of the plant;
wherein the suitability assessment is determined based on the at least one image; and
wherein the acquisition and/or evaluation of the spectral analytical information of the plant is performed in dependence on the suitability assessment.

16. The method according to claim 15, further comprising outputting the suitability assessment via a human-machine interface for visualizing the suitability assessment.

17. The method according to claim 15, wherein the suitability assessment comprises a spatially resolved assessment of the suitability of different positions or areas of the plant for determining, based on the spectral analytical information, nutrient concentrations or a health status.

18. The apparatus according to claim 6, wherein the device for reducing ambient light effects comprises an aperture provided on the housing and projecting outwardly at least partially around a viewing window supported in the housing.

19. The apparatus according to claim 7, wherein the apparatus is configured as a manually holdable apparatus.

20. The apparatus according to claim 11, wherein the at least one evaluation device interface is configured to transmit the suitability assessment to the apparatus according to claim 1 or wherein the evaluation device or the evaluation system is configured to control the at least one evaluation device interface to enable a user selection of the leaf areas to be analyzed.
